(19) United States
(12) Patent Application Publication
(10) Pub. No.: US 2009/0176317 A1
(43) Pub. Date: Jul. 9, 2009

(54) Soluble B7-H1

(75) Inventors: Eugene D. Kwon, Rochester, MN (US); Haidong Dong, Rochester, MN (US); Xavier Frigola Baro, Rochester, MN (US); Brant A. Inman, Chapel Hill, NC (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 12/254,478

(22) Filed: Oct. 20, 2008

Related U.S. Application Data
(63) Continuation-in-part of application No. PCT/US2007/066970, filed on Apr. 19, 2007.
(60) Provisional application No. 60/793,437, filed on Apr. 20, 2006.

(51) Int. Cl. G01N 33/574; G01N 33/566

(52) U.S. Cl. 436/501

(57) ABSTRACT

This document features methods of evaluating mammals by assessing expression of B7-H4 in the vasculature.

### CROSS-REFERENCE TO RELATED APPLICATIONS

This application is a continuation-in-part and claims benefit under 35 U.S.C. § 119(a) of International Application No. PCT/US2007/066970, having an International Filing Date of Apr. 19, 2007, which claims the benefit of priority of U.S. Provisional Application Ser. No. 60/793,437, having a filing date of Apr. 20, 2006, both of which are incorporated herein in their entirety.

### TECHNICAL FIELD

This document relates to a soluble form of B7-H1, and more particularly, to detecting soluble B7-H1 in body fluids to evaluate mammals.

### BACKGROUND

The incidence of renal cell carcinoma (RCC) has increased steadily over the last three decades, and mortality rates continue to rise. See Jemal et al. (2005) CA Cancer J. Clin. 55, 10-30. To date, the only acceptable treatment for clinically localized RCC is surgical extirpation. Improvements in imaging technology have led to a stage migration, and with accompanying surgical advancements, improvements in patient survival have been noted. Pantuck et al. (2001) J. Urol. 166, 1611-1623. Regrettably, the five-year survival of RCC patients is still unacceptably low. This low survival rate reflects the 30% of patients who present with metastatic disease and another 25-30% who will subsequently develop disseminated disease after surgical excision of the primary tumor. Motzer et al. (1996) N. Engl. J. Med. 335, 865-875; and Leibovich et al. (2003) Cancer. 97, 1663-1671. Other treatment modalities for advanced disease such as chemotherapy and radiation have not been shown to be effective. Immunotherapy is one adjunct therapy available, but less than 10% of patients benefit with durable responses. Fyfe et al. (1995) J. Clin. Oncol. 13, 688-696. Limited therapeutic options have done little to improve the median survival of 6-10 months seen in metastatic disease. Figlin et al. (1997) J. Urol. 158, 740-750. Since a large percentage of patients with clinically localized disease subsequently develop metastasis, there is a need for prognostic biomarkers.

### SUMMARY

This document is based in part on the discovery that a soluble form of B7-H1 is present in the serum of cancer patients. Identification of a soluble form of B7-H1 allows expression of B7-H1 to be evaluated in patients by a minimally invasive method. B7-H1 is over-expressed by many human cancers, and impairs anti-tumoral responses by inducing T cell apoptosis and by inhibiting T cell cytokine production, proliferation and cytotoxic function. As such, soluble B7-H1 can be used as a biomarker for diagnosis of cancer and prognosis of a patient with cancer.

In general, this document features a method of evaluating a mammal. The method can comprise, or consist essentially of, (a) providing a body fluid from the mammal, and (b) detecting the presence or absence of B7-H1 in the body fluid. The mammal can be a human. The B7-H1 can be detected immunologically. The B7-H1 can be detected using a monoclonal antibody. The B7-H1 can be detected using a capture antibody and a reporter antibody, where the reporter antibody comprises a label. The label can be a fluorophore, biotin, an enzyme, or a radioisotope. The fluorophore can be fluorescein, fluorescein isothiocyanate (FITC), phycoerythrin (PE), allophycocyanin (APC), or peridinin chlorophyll protein (PerCP). The capture antibody can be attached to a solid substrate. The solid substrate can be selected from the group consisting of a bead and a microtiter plate. The capture antibody can be a polyclonal antibody. The body fluid can be selected from the group consisting of blood, plasma, serum, urine, cerebrospinal fluid, sputum, tears, and saliva. The body fluid can be serum. The mammal can be suspected of having a cancer. The cancer can be renal cell carcinoma. The presence of B7-H1 in the body fluid can indicate the presence of a cancer in the mammal. The presence of B7-H1 in the body fluid can indicate the mammal is more likely to die of the cancer than if B7-H1 is absent.

In another aspect, this document features a method of evaluating a mammal with renal cell carcinoma. The method can comprise, or consist essentially of, (a) providing a body fluid from the mammal, and (b) detecting the presence or absence of B7-H1 in the body fluid. The presence of B7-H1 in the body fluid can indicate the mammal is more likely to die of renal cell carcinoma than if B7-H1 is absent.

In another aspect, this document features a method of detecting B7-H1 in a body fluid. The method can comprise, or consist essentially of, (a) providing a solid substrate, the solid substrate coated with capture antibodies having binding affinity for soluble B7-H1; (b) contacting the body fluid with the solid substrate under conditions in which soluble B7-H1, if present, becomes bound to the solid substrate to form a first reacted solid substrate; (c) contacting the first reacted solid substrate with a reporter antibody having binding affinity for soluble B7-H1 to form a second reacted solid substrate; and (d) detecting the presence or absence of the reporter antibody on the second reacted solid substrate, where the presence of reporter antibody indicates that soluble B7-H1 is present in the body fluid. The reporter antibody can comprise a label selected from the group consisting of a radioisotope, a fluorophore, a luminescent moiety, biotin, and an enzyme. Detecting the presence or absence of the reporter antibody can comprise contacting the second reacted solid substrate with a secondary antibody having binding affinity for the reporter antibody, where the secondary antibody comprises a label. Detecting the presence or absence of the reporter antibody can comprise contacting the second reacted solid substrate with a reagent having binding affinity for the reporter antibody, where the reagent comprises a label. The solid substrate can be a bead or a microtiter plate.

In another aspect, this document features a kit for detecting soluble B7-H1. The kit can comprise, or consist essentially of, a pair of antibodies, each antibody of the pair having binding affinity for soluble B7-H1, where each antibody of the pair recognizes a different epitope of soluble B7-H1. The kit can further comprise a solid substrate, a positive control, and/or a negative control.

In another aspect, this document features a method for determining whether a mammal has cancer. The method can comprise, or consist essentially of, (a) determining whether or not a mammal has a body fluid containing an elevated level of a B7-H1 polypeptide, and (b) classifying the mammal as having cancer if the mammal has the elevated level and classifying the mammal as not having cancer if the mammal does not have the elevated level. The mammal can be a human. The body fluid can be blood, serum, plasma, or urine. The cancer can be renal cell carcinoma.

In another aspect, this document features a method for assessing the effectiveness of a cancer treatment. The method can comprise, or consist essentially of, determining whether or not a mammal having cancer and having received a treatment for the cancer has a level of a B7-H1 polypeptide that is lower than that observed prior to the treatment, and classifying the cancer treatment as being effective if the level of a B7-H1 polypeptide is lower than that observed prior to the treatment, or classifying the cancer treatment as not being effective if the level of a B7-H1 polypeptide is not lower than that observed prior to the treatment.

In another aspect, this document features a method for determining whether a mammal has cancer that has progressed. The method can comprise, or consist essentially of, determining whether or not a level of a B7-H1 polypeptide in a body fluid of a mammal increases over time, and classifying the mammal as having cancer that has progressed if the level of a B7-H1 polypeptide has increased, or classifying the mammal as not having cancer that has progressed if the level of B7-H1 polypeptide has not increased.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention will be apparent from the following description, from the drawings and from the claims.

### BRIEF DESCRIPTION OF DRAWINGS

FIG. 1 contains the predicted amino-acid sequence of human B7-H1 (SEQ ID NO:1), indicating the predicted signal peptide, immunoglobulin V-like (Ig-V-like) domain, immunoglobulin C-like (Ig-C-like) domain, transmembrane (TM) region, and potential N-linked glycosylation sites (*).

FIG. 2 is a graph demonstrating that B7-H1 was detected in the serum of 25% of RCC patients (1/48) and 8% of normal donors (1/12). All samples were diluted 1:20 in PBS. B7-H1 fusion polypeptide and human IgG Fc polypeptide were used as positive and negative controls, respectively.

FIG. 3 is a graph plotting B7-H1 polypeptide levels in serum samples from 14 normal donors (DONOR) and 65 clear cell renal cell carcinoma patients (RCC) analyzed using a sandwich ELISA with anti-B7-H1 antibodies. The horizontal bars represent median values. The p-value of the Wilcoxon rank sum test was 0.0008.

FIG. 4A is histogram plot of flow cytometry data obtained by analyzing cells positive for B7-H1 polypeptide expression that were stained with a biotinylated monoclonal anti-B7-H1 antibody, 2.2B, in the presence (dotted line) or absence (solid line) of an unbiotinylated monoclonal anti-B7-H1 antibody, 5H1-A3. The filled histogram is a plot of data obtained by analyzing unbiotinylated control cells. FIG. 4B is a histogram plot of flow cytometry data obtained by analyzing cells positive for B7-H1 polypeptide expression that were stained with a biotinylated monoclonal anti-B7-H1 antibody, 5H1-A3, in the presence (dotted line) or absence (solid line) of an unbiotinylated monoclonal anti-B7-H1 antibody, 2.2B. The filled histogram is a plot of data obtained by analyzing unbiotinylated control cells.

FIG. 5 is a histogram plot of flow cytometry data obtained by analyzing cells positive for B7-H1 polypeptide expression that were stained using biotinylated or unbiotinylated 5H1-A3 or 2.2B monoclonal anti-B7-H1 antibody. un-5H1=unbiotinylated 5H1-A3 antibody, un-2.2B=unbiotinylated 2.2B antibody, b-5H1=biotinylated 5H1-A3 antibody, and b-2.2B=biotinylated 2.2B antibody.

FIG. 6 is a graph plotting levels of B7-H1 polypeptide in cell culture supernatants incubated with BT10B (BT 10B SN), HMCI (HMCI SN), or Caki2 (Caki2 SN) cells for three to four days. The B7-H1 polypeptide levels were analyzed using an ELISA assay. Recombinant human B7-H1 fusion polypeptide (B7H1Fc) was used as a positive control. Phosphate-buffered saline (PBS), recombinant human P-selectin fusion polypeptide (PSelFc), and fresh cell culture media for BT10B and HMCI cells (BT media and HMCI media, respectively), that had not been incubated with cells, were negative controls.

FIG. 7 is a graph plotting levels of B7-H1 polypeptide in cell culture supernatants from 624MEL, B7H1/624MEL, J82, Caki-2, BT10B, and BT10C cells. The B7-H1 polypeptide levels were measured using an ELISA assay. Recombinant human B7-H1 fusion polypeptide (B7-H1Fc) was used as a positive control. PBS and fresh cell culture media for each cell type, which had not been incubated with cells, were analyzed as negative controls.

FIG. 8 is a Western blot analyzing B7-H1 polypeptide expression in extracts from 624MEL, B7-H1/624MEL, BT10B, BT10C, and Caki-2 cells. Immunoblotting was performed using biotinylated monoclonal anti-B7-H1 antibody 5H1-A3. The membrane was stripped and reprobed for actin polypeptide as a loading control.

FIG. 9A is a graph plotting the calibration of an ELISA system to measure sB7-H1. The graph depicts results from three separate ELISA experiments in which serial dilutions of hB7-H1 -Fc were tested four to six times and fitted into a 4-parameter logistic regression model. The black line denotes the fitted model; the darker gray area adjacent to the line denotes the 95% CI; and the lighter gray area denotes the 95% prediction interval. FIG. 9B is a graph plotting serum B7-H1 concentrations in the sera of ccRCC patients (n=58) compared with normal controls (n=80); p<0.001, as determined by ELISA. FIG. 9C is a graph plotting sB7-H1 levels in human cancer cell lines, as determined by ELISA. Open bars, control media; hatched bars, media from B7-H1− cell lines; solid gray bars, media from other B7-H1+ cell lines. FIG. 9D is a pair of histograms plotting apoptosis rates in purified and activated CD4+ T cells (left panel) and purified and activated CD8+ T cells (right panel) in response to solubilized hB7-H1-Fc, as determined by Annexin V staining. Solid line in left panel, hB7-H1-Fc; dashed line in left panel, P-Selectin-Fc control (p=0.019). Purified and activated CD8+ T cells were not differentially affected by exposure to solubilized hB7-H1-Fc versus hP-Selectin-Fc control (p=0.899). Figures are representative of cytometric measurements for Annexin V staining conducted on II different patient samples. Shaded areas represent Annexin V staining for control cells not exposed to solubilized protein.

FIG. 10A is an alignment showing the predicted amino acid sequence of splice-variant sB7-H1^{Δ531-636} (SEQ ID NO:2) as compared to the sequence of canonical full-length B7-H1 (SEQ ID NO:1). Predicted domains are identified below the amino acid sequences. Identical amino acids are shaded. FIG. 10B is a structural alignment of hB7-H1 and sB7-H1^{Δ531-636}, indicating that the sB7-H1 splice variant lacks a portion of its IgV-C domain as well as its complete TM and intracellular domains. Each box illustrates exon regions (E) that are either translated (shaded) or untranslated (open).

FIG. 10C shows amino acid sequences from the regions of canonical B7-H1 and sB7-H1^{Δ531-636} at which the splice variation occurs. FIG. 10D is a picture of a gel containing cDNA obtained from RT-PCR amplification of mRNA extracted from BT10B, DU-145 and Caki-2 cancer cell lines. The 651 bp band corresponds to full-length B7-H1, and the 547 bp band corresponds to sB7-H1^{Δ531-636} (top panel). Additional RT-PCR samples using a different pair of primers specific for the splice variant yielded the single anticipated 490 bp product corresponding to sB7-H1^{Δ531-636} (bottom panel). β-actin was used as a loading control (middle panel).

FIG. 11A is a picture of a gel containing cDNA obtained by RT-PCR amplification of mRNA extracted from digested and ccRCC tumor cell-enriched specimens. The 490 bp product from sB7-H1^{Δ531-636} was detected in four of six samples tested (top panel). β-actin was used as a loading control (bottom panel). FIG. 11B is a picture of representative ccRCC tumor specimens with both membranous (left panel) and cytoplasmic-only (right panel) B7-H1+IHC staining. FIG. 11C is a Kaplan-Meier plot for patients with cytoplasmic-only B7-H1+ tumors, patients with membranous B7-H1+ tumors (without cytoplasmic-only subsets of cells), and patients with B7-H1-negative tumors.

FIG. 12 is a graph indicating specificity of the B7-H1 ELISA assay. The ELISA was highly specific for human B7-H1, and did not cross-react with other B7 family members (including B7-H2, B7-H3, B7-H4, CD80 and PD-1) or irrelevant control proteins (including P-Selectin and mouse IgG). The results of three different ELISA experiments with four to six replicates each are depicted.

FIG. 13 is a graph plotting B7-H1 levels in the sera of pancreatic cancer patients (n=19) and non-cancer patients (n=95), p<0.001. Means and 95% CI for each group are also depicted.

FIG. 14 is a series of histograms plotting expression of PD-1 on activated T cells. Purified human CD4 (FIG. 14A) and CD8 (FIG. 14B) T cells were activated with anti-CD3 and analyzed for PD-1 expression after 1 day (left column) or 3 days (right column). Percentages of positive cells were obtained after subtracting the isotype background (filled histograms).

FIG. 15 is a series of photographs showing cytoplasmic-only B7-H1 staining in other malignancies. A cytoplasmatic-only staining pattern, without a membranous component, was found in esthesioneuroblastoma (FIG. 15A), medullary carcinoma of thyroid (FIG. 15B), paraganglioma (FIG. 15C), and ovarian serous carcinoma (FIG. 15D).

### DETAILED DESCRIPTION

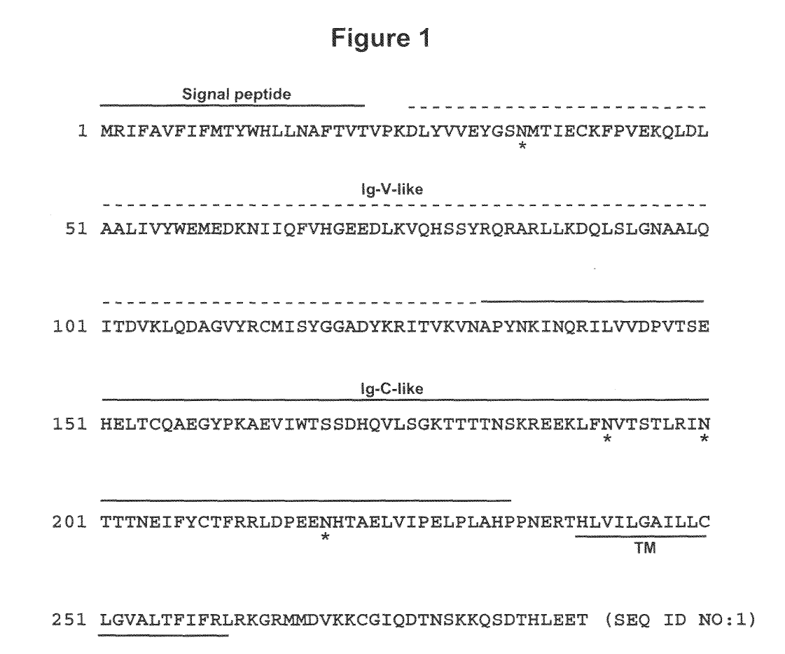

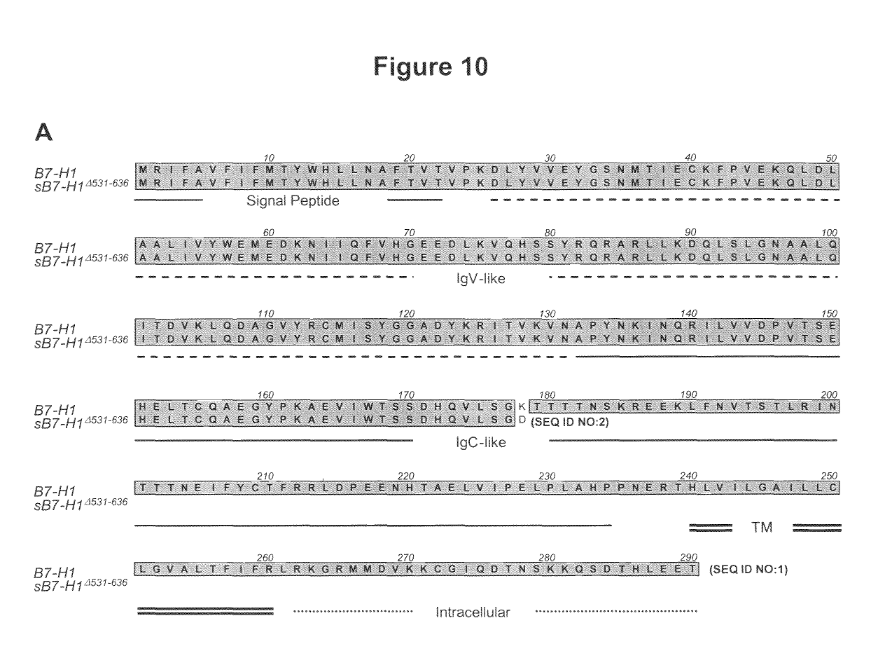

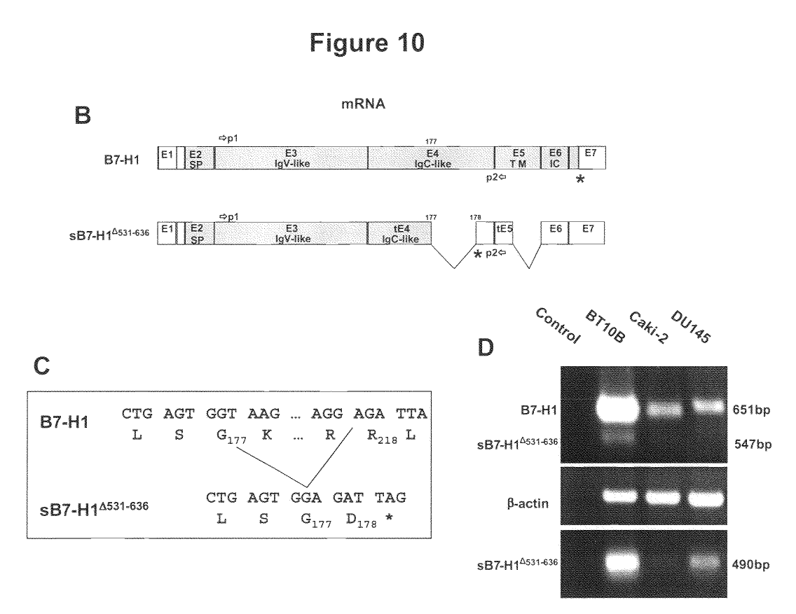

In general, this document provides methods and materials for evaluating mammals for the presence, absence, or amount of B7-H1 in a body fluid (e.g., blood, plasma, serum, urine, cerebrospinal fluid, sputum, tears, or saliva). As used herein, the term “B7-H1” refers to B7-H1 from any mammalian species and the term “hB7-H1” refers to human B7-H1. Further details on B7-H1 polypeptides and nucleic acids are provided in U.S. Pat. No. 6,803,192 and co-pending U.S. application Ser. No. 09/649,108, the disclosures of which are incorporated herein by reference in their entirety. The nucleotide and amino acid sequences of hB7-H1 can be found in GenBank under Accession Nos. AF177937 (GI:6708118) and AAF25807 (GI:6708119), respectively. A reference amino acid sequence for hB7-H1 (SEQ ID NO:1) also is shown in FIGS. 1 and 10A herein. B7-H1 (also known as PD-L1) is a glycosylated membrane polypeptide of the B7 costimulatory family. The open reading frame of the B7-H1 gene encodes a type I transmembrane polypeptide of 290 amino acids, consisting of immunoglobulin V-like and C-like domains, a hydrophobic transmembrane domain and a cytoplasmic tail of 30 amino acids (FIG. 1). The sequence reveals four structural cysteines, which are involved in the formation of disulfide bonds of the immunoglobulin V-like and C-like domains. As disclosed herein, however, a soluble form of B7-H1 (e.g., B7-H1 lacking all or part of the transmembrane domain and/or all or part of the cytoplasmic tail) can be detected in body fluids such as serum.

B7-H1 is a negative regulator of T cell-mediated immunity. See, Dong et al. (1999) Nat. Med. 5, 1365-1369; Dong et al. (2002) Nat. Med. 8, 793-800; and Thompson et al. (2004) Proc. Natl. Acad. Sci. USA 101, 17174-17179. This molecule is constitutively expressed on macrophage-lineage cell surfaces and is expressed in multiple human malignancies. B7-H1 is normally expressed in very limited amounts by monocyte-lineage cells within the liver, lung and tonsils. B7-H1 is markedly over-expressed by many human cancers and has been shown to impair anti-tumoral responses by inducing T cell apoptosis and by inhibiting T cell cytokine production, proliferation and cytotoxic function. As such, B7-H1 expression by tumor cells may be a potent contributor to the immunosuppressive profile that is typically exhibited by advanced cancer patients.

### Methods of Evaluating Mammals

In general, methods of the invention include detecting the presence, absence, or amount of B7-H1 in a body fluid of a subject. In some embodiments, the amount of B7-H1 in a body fluid can be expressed relative to the amount from a control population (e.g., the average amount of B7-H1 from a plurality of subjects without cancer). Suitable subjects can be mammals, including, for example, humans (e.g., patients suspected of having a cancer), non-human primates such as monkeys, baboons, or chimpanzees, horses, cows (or oxen or bulls), pigs, sheep, goats, cats, rabbits, guinea pigs, hamsters, rats, gerbils, and mice.

As described herein, soluble B7-H1 was detected in the serum of at least 25% of the RCC patients examined. In contrast, soluble B7-H1 was detected in the serum of only 8% of normal control subjects. Since B7-H1 is markedly over-expressed by many human cancers, impairs anti-tumoral responses, and in RCC patients, is associated with aggressive tumors and increased risk for succumbing to death due to RCC, detecting soluble B7-H1 in a body fluid can be used as a biomarker for diagnosing cancer, determining prognosis, or assessing risk of cancer progression. For example, the presence of soluble B7-H1 in a body fluid from a mammal (e.g., a patient suspected of having a cancer) can indicate the presence of a cancer in the mammal. The presence of soluble B7-H1 in a mammal diagnosed with cancer also can indicate that the mammal is more likely to die of the cancer than if B7-H1 is absent. Since a number of cancers express B7-H1, the methods of the invention can be used to evaluate mammals that are suspected of having a variety of cancers, including, for example, renal cancer, hematological cancer (e.g., leukemia or lymphoma), neurological cancer, melanoma, breast cancer, lung cancer, head and neck cancer, gastrointestinal cancer, liver cancer, pancreatic cancer, genitourinary cancer, bone cancer, or vascular cancer. Methods of the invention are particularly useful for evaluating mammals with RCC, lung, ovarian, and colon cancer. Additional factors that can be considered when evaluating a mammal can include, for example, patient history, family history, genetic factors, overall health of the mammal, and/or previous responses to therapy.

Furthermore, detecting the presence, absence, or amount of B7-H1 in a body fluid can be used to provide valuable clues as to the course of action to be undertaken in treatment of the cancer since the presence of B7-H1 can indicate a particularly aggressive course of cancer. Detecting the presence, absence, or amount of B7-H1 in a body fluid also can be used to monitor the response of a mammal to a cancer therapy. In some cases, detecting the presence, absence, or amount of B7-H1 in body fluids can be used in population screening for cancer.

In some cases, a mammal can be classified as having cancer if it is determined that a body fluid from the mammal contains a detectable level of a B7-H1 polypeptide. In some cases, a mammal can be classified as not having cancer if it is determined that a body fluid from the mammal does not contain a detectable level of a B7-H1 polypeptide. In some cases, a mammal can be classified as having cancer if it is determined that a body fluid (e.g., blood) from the mammal contains an elevated level of a B7-H1 polypeptide. If the level of a B7-H1 polypeptide in a body fluid from a mammal is not elevated, then the mammal can be classified as not having cancer. The term “elevated level” as used herein with respect to a level of a B7-H1 polypeptide is any level that is greater than a reference level for the B7-H1 polypeptide. The term “reference level” as used herein with respect to a B7-H1 polypeptide is the level of the B7-H1 polypeptide typically expressed by mammals free of cancer. For example, a reference level of a B7-H1 polypeptide can be the median level of the B7-H1 polypeptide that is present in samples obtained from a random sampling of humans that are free of cancer. Control samples used to determine a reference level can be obtained from any appropriate number of mammals (e.g., 10, 20, 30, 40, 50, 75, 100, 125, 150, 175, 200, 250, 300, 400, 500, 600, 700, 800, 900, 1000 or more mammals) from the same species as the mammal being evaluated. In some cases, control samples can be obtained from humans of the same race, age group, and/or geographic location as the mammal being evaluated.

It will be appreciated that levels from comparable samples are used when determining whether or not a particular level of a B7-H1 polypeptide is an elevated level. For example, the median level of a B7-H1 polypeptide present in serum from a random sampling of mammals may be X units/g of serum, while the median level of a B7-H1 polypeptide present in urine may be Y units/g of urine. In this case, the reference level for a B7-H1 polypeptide in serum would be X units/g of serum, and the reference level for a B7-H1 polypeptide in urine would be Y units/g of urine. Thus, when determining whether or not the level of a B7-H1 polypeptide in serum is elevated, the measured level would be compared to the reference level in serum. In addition, a level of a B7-H1 polypeptide in a body fluid from a mammal is typically compared to a reference level determined by analyzing samples using a technique comparable to the technique used to measure the B7-H1 level in the mammal being evaluated.

An elevated level of a B7-H1 polypeptide can be any level provided that the level is greater than a corresponding reference level. For example, an elevated level of a B7-H1 polypeptide can be 1.1, 1.2, 1.3, 1.4, 1.5, 1.6, 1.7, 1.8, 1.9, 2.0, 2.2, 2.4, 2.6, 2.8, 3.0, 3.3, 3.5, 3.7, 4.0, 4.5, 5.0, 6.1, 7.2, 8.0, 9.1, 10.0, 15.5, 20.7, or more times greater than a reference level for the B7-H1 polypeptide. In some cases, an elevated level of a B7-H1 polypeptide can be a level that is at least 2 percent (e.g., at least 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500 percent) greater than a corresponding reference level. In addition, a reference level can be any amount. For example, a reference level for a B7-H1 polypeptide can be zero. In this case, any level of the B7-H1 polypeptide greater than zero would be an elevated level.

This document also provides methods and materials for determining the prognosis of a mammal having cancer (e.g., RCC). For example, the presence of a B7-H1 polypeptide in a body fluid from a mammal having cancer can indicate that the mammal is susceptible to a poor outcome, and the absence of a B7-H1 polypeptide in a body fluid from a mammal having cancer can indicate that the mammal is susceptible to a good outcome. In some cases, the presence of a detectable amount of a B7-H1 polypeptide in a body fluid of a mammal having cancer can indicate that the mammal is susceptible to a poor outcome, and the absence of a detectable amount of a B7-H1 polypeptide in a body fluid from a mammal can indicate that the mammal is susceptible to a good outcome. In some cases, the presence of an elevated level of a B7-H1 polypeptide in a body fluid from a mammal having cancer can indicate that the mammal is susceptible to a poor outcome, and the absence of an elevated level of a B7-H1 polypeptide in a body fluid from a mammal having cancer can indicate that the mammal is susceptible to a good outcome.

The prognosis of a mammal having cancer also can be correlated with the degree of elevation of a B7-H1 polypeptide in a body fluid from a mammal. For example, a greater degree of elevation of a B7-H1 polypeptide level in a body fluid from a mammal above a corresponding reference level can indicate that the mammal is more susceptible to a poor outcome, and a lesser degree of elevation of a B7-H1 polypeptide level in a body fluid from a mammal above a corresponding reference level can indicate that the mammal is less susceptible to a poor outcome. In some cases, a level of a B7-H1 polypeptide in a body fluid from a mammal that is at least one standard deviation higher than a reference level can indicate that the mammal is more susceptible to a poor outcome than a mammal having a level of the B7-H1 polypeptide in a corresponding body fluid that is less than one standard deviation higher than the reference level.

In some cases, the presence, absence, or level of a B7-H1 polypeptide in a body fluid from a mammal can be used in combination with other factors to determine the prognosis of a mammal having cancer. For example, the presence, absence, or level of a B7-H1 polypeptide in a body fluid from a mammal having cancer can be used in combination with the clinical stage of the cancer, results of a physical examination, information about a family history of cancer, and/or results from imaging (e.g., magnetic resonance imaging) to determine whether or not the mammal is likely to have a poor outcome. A mammal that is susceptible to a poor outcome can have a more aggressive cancer, experience more rapid cancer progression, and/or die sooner of cancer than a mammal that is susceptible to a good outcome. Information about the prognosis of a mammal having cancer can be used to guide treatment selection. For example, a mammal identified as being susceptible to a poor prognosis can be treated earlier and more aggressively than a mammal identified as being susceptible to a good outcome.

Once a mammal has been identified as having cancer, the mammal can be subsequently evaluated or monitored over time for progression of the cancer. For example, a mammal can be classified as having a cancer that has progressed if it is determined that a body fluid from the mammal contains a B7-H1 polypeptide at a level that is greater than the level of the B7-H1 polypeptide observed in a corresponding body fluid obtained previously from the mammal. In some cases, a mammal can be classified as having a cancer that has not progressed if it is determined that a body fluid from the mammal contains a B7-H1 polypeptide at a level that is equal to or less than the level of the B7-H1 polypeptide observed in a corresponding body fluid obtained previously from the mammal.

A mammal that has been treated for cancer can be monitored for recurrence of the cancer. For example, a mammal that has been treated for cancer can be classified as having a recurring cancer if it is determined that a body fluid taken from the mammal after treatment (e.g., surgical resection of a tumor) contains a B7-H1 polypeptide at a level that is greater than the level of the B7-H1 polypeptide observed in a corresponding body fluid obtained from the mammal at an earlier time point after treatment. In some cases, a mammal can be classified as not having a recurring cancer if it is determined that a body fluid taken from the mammal after treatment with a cancer therapy contains a B7-H1 polypeptide at a level that is equal to or less than the level of the B7-H1 polypeptide observed in a corresponding body fluid obtained from the mammal at an earlier time point after treatment. A mammal can be monitored for progression or recurrence of a cancer over any period of time with any frequency. For example, a mammal can be monitored once a year, twice a year, three times a year, or more frequently. In some cases, a mammal can be monitored every three months for five years, or once a year for as long as the mammal is alive.

Methods and materials provided herein also can be used to determine whether or not a cancer therapy is effective. For example, a level of a B7-H1 polypeptide can be determined in a body fluid taken from a mammal prior to treatment with a cancer therapy, and the level can be compared to a level of the B7-H2 polypeptide in a corresponding body fluid taken from a mammal during or after treatment. A decrease in the level of the B7-H1 polypeptide in the fluid taken during or after treatment as compared to the level in the fluid taken before treatment can indicate that the treatment is effective. In some cases, an increase or no change in the level of the B7-H1 polypeptide in a body fluid taken during or after a cancer treatment as compared to the level in a corresponding fluid taken before treatment can indicate that the treatment is not effective. In some cases, a decrease in a level of a B7-H1 polypeptide in a body fluid taken from a mammal during or after a cancer treatment as compared to the level in a corresponding fluid taken at an earlier time point during treatment can indicate that the treatment is effective. In some cases, an increase or no change in a level of a B7-H1 polypeptide in a body fluid taken from a mammal during or after a cancer treatment as compared to the level in a corresponding fluid taken at an earlier time point during treatment can indicate that the treatment is not effective.

Any appropriate method can be used to obtain a body fluid from a mammal for analysis of the presence, absence, or amount of a B7-H1 polypeptide. For example, a blood sample can be obtained by peripheral venipuncture, and urine samples can be obtained using standard urine collection techniques. Once obtained, a body fluid can be manipulated prior to being analyzed. For example, a body fluid can be centrifuged prior to being analyzed for a B7-H1 polypeptide. In some cases, a blood sample can be allowed to clot and can be centrifuged prior to being analyzed. In some cases, a body fluid can be stored (e.g., at 4° C.) prior to being analyzed for a B7-H1 polypeptide. In addition, polypeptides can be extracted from a body fluid and can be fractionated (e.g., on a column or in a gel) prior to being analyzed for a B7-H1 polypeptide.

Typically, the presence, absence, or amount of B7-H1 in the body fluid is determined by detecting soluble B7-H1 polypeptide. Methods of detecting polypeptides in body fluids are known in the art. For example, antibodies that bind to an epitope specific for soluble B7-H1 can be used to detect B7-H1 in body fluid. As used herein, the terms “antibody” or “antibodies” include intact molecules (e.g., polyclonal antibodies, monoclonal antibodies, humanized antibodies, or chimeric antibodies) as well as fragments thereof (e.g., single chain Fv antibody fragments, Fab fragments, and F(ab)₂ fragments) that are capable of binding to an epitopic determinant of B7-H1 (e.g., hB7-H1). The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). Polyclonal antibodies are heterogeneous populations of antibody molecules that are contained in the sera of the immunized animals. Monoclonal antibodies are homogeneous populations of antibodies to a particular epitope of an antigen. An antibody directed against a B7-H1 polypeptide can bind the polypeptide with an affinity of at least 10⁴ mol⁻¹ (e.g., at least 10⁵, 10⁶, 10⁷, 10⁸, 10⁹, 10¹⁰, 10¹¹, or 10¹² mol⁻¹).

Antibody fragments that can bind to B7-H1 can be generated by known techniques. For example, F(ab′)₂ fragments can be produced by pepsin digestion of the antibody molecule; Fab fragments can be generated by reducing the disulfide bridges of F(ab′)₂ fragments. Alternatively, Fab expression libraries can be constructed. See, for example, Huse et al., Science, 246:1275 (1989). Once produced, antibodies or fragments thereof are tested for recognition of B7-H1 by standard immunoassay methods including ELISA techniques, radioimmunoassays, and Western blotting. See, Short Protocols in Molecular Biology, Chapter 11, Green Publishing Associates and John Wiley & Sons, Edited by Ausubel, F. M et al., 1992.

Antibodies having specific binding affinity for B7-H1 can be produced through standard methods. See, for example, Dong et al. (2002) Nature Med. 8:793-800. In general, a B7-H1 polypeptide (e.g., B7-H1 comprising or consisting of the extracellular domain of B7-H1) can be recombinantly produced, or can be purified from a biological sample, and used to immunize animals. As used herein, the term “polypeptide” refers to a polypeptide of at least five amino acids in length. To produce a recombinant B7-H1 polypeptide, a nucleic acid sequence encoding the appropriate polypeptide can be ligated into an expression vector and used to transform a bacterial or eukaryotic host cell. Nucleic acid constructs typically include a regulatory sequence operably linked to a B7-H1 nucleic acid sequence. Regulatory sequences do not typically encode a gene product, but instead affect the expression of the nucleic acid sequence. In bacterial systems, a strain of Escherichia coli such as BL-21 can be used. Suitable E. coli vectors include without limitation the pGEX series of vectors that produce fusion polypeptides with glutathione S-transferase (GST). Transformed E. coli are typically grown exponentially, then stimulated with isopropylthiogalactopyranoside (IPTG) prior to harvesting. In general, such fusion polypeptides are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

Mammalian cell lines that stably express a B7-H1 polypeptide can be produced by using expression vectors with the appropriate control elements and a selectable marker. For example, the eukaryotic expression vector pcDNA.3.1+ (Invitrogen, San Diego, Calif.) can be used to express a B7-H1 polypeptide in, for example, COS cells, Chinese hamster ovary (CHO), human melanoma cells, or HEK293 cells. Following introduction of the expression vector by electroporation, DEAE dextran, or other suitable method, stable cell lines can be selected. Alternatively, B7-H1 can be transcribed and translated in vitro using wheat germ extract or rabbit reticulocyte lysate.

In eukaryotic host cells, a number of viral-based expression systems also can be utilized to express a B7-H1 polypeptide. A nucleic acid encoding a B7-H1 polypeptide can be introduced into a SV40, retroviral or vaccinia based viral vector and used to infect host cells. Alternatively, a nucleic acid encoding a B7-H1 polypeptide can be cloned into, for example, a baculoviral vector and then used to transfect insect cells.

Various host animals can be immunized by injection of the B7-H1 polypeptide. Host animals include rabbits, chickens, mice, guinea pigs, and rats. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a B7-H1 polypeptide and standard hybridoma technology. In particular, monoclonal antibodies can be obtained by any technique that provides for the production of antibody molecules by continuous cell lines in culture such as described by Kohler et al., Nature, 256:495 (1975), the human B-cell hybridoma technique (Kosbor et al., Immunology Today, 4:72 (1983); Cole et al., Proc. Natl. Acad. Sci. USA, 80:2026 (1983)), and the EBV-hybridoma technique (Cole et al., “Monoclonal Antibodies and Cancer Therapy”, Alan R. Liss, Inc., pp. 77-96 (1983)). Such antibodies can be of any immunoglobulin class including IgG, IgM, IgE, IgA, IgD, and any subclass thereof. The hybridoma producing the monoclonal antibodies of the invention can be cultivated in vitro and in vivo.

In immunological assays, an antibody having specific binding affinity for B7-H1 or a secondary antibody that binds to such an antibody can be labeled, either directly or indirectly. Suitable labels include, without limitation, radioisotopes (e.g., ¹²⁵I, ¹³¹I, ³⁵S, ³H, ³²P, ³³P, or ¹⁴C), fluorophores (e.g., fluorescein, fluorescein-5-isothiocyanate (FITC), PerCP, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting B7-H1 can be performed in a variety of known formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. Methods of detecting B7-H1 generally include contacting a body fluid with an antibody that binds to B7-H1 and detecting or quantifying binding of B7-H1 to the antibody. For example, an antibody having specific binding affinity for B7-H1 can be immobilized on a solid substrate by any of a variety of methods known in the art and then exposed to the biological sample. Binding of B7-H1 to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden). Alternatively, the antibody can be labeled and detected as described above. A standard curve using known quantities of B7-H1 can be generated to aid in the quantitation of B7-H1 levels.

In other embodiments, a “sandwich” assay in which a capture antibody is immobilized on a solid substrate is used to detect the presence, absence, or amount of soluble B7-H1. The solid substrate can be contacted with the biological sample such that any B7-H1 in the sample can bind to the immobilized antibody. The presence of B7-H1 bound to the antibody can be determined using a “reporter” antibody having specific binding affinity for B7-H1 and the methods described above. It is understood that in these sandwich assays, the capture antibody should not bind to the same epitope (or range of epitopes in the case of a polyclonal antibody) as the reporter antibody. Thus, if a monoclonal antibody is used as a capture antibody, the reporter antibody can be another monoclonal antibody that binds to an epitope that is either completely physically separated from or only partially overlaps with the epitope to which the capture monoclonal antibody binds, or a polyclonal antibody that binds to epitopes other than or in addition to that to which the capture monoclonal antibody binds. If a polyclonal antibody is used as a capture antibody, the reporter antibody can be either a monoclonal antibody that binds to an epitope that is either completely physically separated from or partially overlaps with any of the epitopes to which the capture polyclonal antibody binds, or a polyclonal antibody that binds to epitopes other than or in addition to that to which the capture polyclonal antibody binds.

Suitable solid substrates to which an antibody (e.g., a capture antibody) can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be particularly useful when an automated immunoassay system is used.

Alternative techniques for detecting soluble B7-H1 include mass-spectrophotometric techniques such as electrospray ionization (ESI), liquid chromatography-mass spectrometry (LC-MS), and matrix-assisted laser desorption-ionization (MALDI). See, for example, Gevaert et al., Electrophoresis 22(9):1645-51, 2001; Chaurand et al., J Am Soc Mass Spectrom 10(2):91-103, 1999. Mass spectrometers useful for such applications are available from Applied Biosystems (Foster City, Calif.); Bruker Daltronics (Billerica, Mass.) and Amersham Pharmacia (Sunnyvale, Calif.). Arrays for detecting polypeptides, two-dimensional gel analysis, and chromatographic separation techniques also can be used to detect soluble B7-H1 polypeptide.

This document also provides methods and materials to assist medical or research professionals in determining whether or not a mammal has cancer, and whether or not a mammal having cancer is susceptible to a poor outcome. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the presence, absence, or level of a B7-H1 polypeptide in a body fluid from a mammal, and (2) communicating information about that level to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

### Articles of Manufacture

Antibodies that can bind to a soluble B7-H1 polypeptide (e.g., soluble hB7-H1) can be combined with packaging material and sold as a kit for detecting B7-H1 from body fluid, diagnosing a cancer, determining prognosis of a subject with cancer, or determining risk of cancer progression in a subject. For example, a kit can include a pair of antibodies, where each antibody of the pair has binding affinity for B7-H1 and where each antibody recognizes a different epitope of soluble B7-H1. Components and methods for producing articles of manufactures are well known. In addition, the articles of manufacture may further include reagents such as secondary antibodies, sterile water, pharmaceutical carriers, buffers, indicator molecules, solid substrates (e.g., beads, microtiter plate), and/or other useful reagents (e.g., a positive control such as B7-H1 fusion polypeptide in which the extracellular domain of B7-H1 is fused to the CH2-CH3 domain of mouse immunoglobulin G2a and/or a negative control such as human IgG Fc polypeptide) for detecting B7-H1 from body fluids, diagnosing a cancer, determining prognosis of a subject with cancer, or determining risk of cancer progression in a subject. The antibodies can be in a container, such as a plastic, polyethylene, polypropylene, ethylene, or propylene vessel that is either a capped tube or a bottle. In some embodiments, the antibodies can be included on a solid substrate such as bead, microtiter plate, or a handheld device for bedside testing. Instructions describing how the various reagents are effective for detecting B7-H1 from body fluids, diagnosing a cancer, determining prognosis of a subject with cancer, or determining risk of cancer progression in a subject also may be included in such kits.

The invention will be further described in the following examples, which do not limit the scope of the invention described in the claims.

### EXAMPLES

### Example 1

### Generation of Anti-B7-H1 Monoclonal Antibodies for ELISA

A plasmid containing a full-length B7-H1 coding sequence (pcDNA3-B7-H1) was transfected into human melanoma 624mel cells by the calcium phosphate method, and cells were selected for resistance to G418. BALB/c mice that were seven to eight weeks old were immunized with human melanoma cells transduced with the plasmid containing a full-length B7-H1 coding sequence (5×10⁶ cells per mouse, i.p.). The mice were immunized twice weekly over a five to six week period. Lymphocytes were subsequently isolated and fused with A38 cells to form a hybridoma using standard techniques. Hybridoma supernatants were screened by ELISA for reactivity against a B7-H1.huFc fusion polypeptide. The B7-H1.huFc fusion polypeptide, also referred to as B7-H1hIgG, was prepared by fusing a nucleic acid encoding the extracellular domain of a human B7-H1 polypeptide to a nucleic acid encoding the CH2-CH3 domain of a human immunoglobulin G in an expression plasmid. Hybridoma supernatants also were screened for the absence of cross-reactivity to an irrelevant polypeptide. B7-H1 polypeptide-specific hybridoma cells were cloned and hybridoma clone 2.2B was selected.

Seven to eight week old BALB/c mice were immunized with a human B7-H1mIgG2A fusion polypeptide. The human B7-H1mIgG2A fusion polypeptide was prepared by fusing a nucleic acid encoding the extracellular domain of a human B7-H1 polypeptide to a nucleic acid encoding the CH2-CH3 domain of a mouse immunoglobulin G2a in an expression plasmid. The mice were injected subcutaneously with 100 μg of the B7-H1mIgG2A fusion polypeptide mixed with Freund's adjuvant. Injections were administered twice weekly over a five to six week period.

Hybridoma supernatants were screened by ELISA for reactivity against B7-H1.huFc polypeptide and for the absence of cross-reactivity to an irrelevant polypeptide. B7-H1 polypeptide-specific hybridoma cells were cloned, and clone 5H1-A3 was selected. The two clones of monoclonal antibody (mAb), 2.2B and 5H1-A3, recognize different antigen epitopes (see Example 4). In ELISA assays, mAb 2.2B was used as a capture antibody and mAb 5H1-A3 was used as a detecting antibody.

### Example 2

### Sandwich ELISA Detection for B7-H1

A sandwich ELISA assay was created using mAb 2.2B and 5H1-A3 to determine if a soluble form of B7-H1 polypeptide was present in serum. Monoclonal antibody 2.2B was used as a coating antibody, whereas biotinylated mAb 5H1-A3 was used as a detection antibody. After each step, assay plates were washed three times with Washing Buffer (PBS with 0.05% Tween-20) using a microplate washer (Bio-Tek, Winooski, Vt.). High-binding polystyrene plates (Corning Life Sciences, Bedford, Mass.) were coated overnight at 4° C. with 0.1 μg/well of anti-B7-H1 mAb. The coating solution was aspirated off, the plates were washed, and free binding sites were blocked with 200 μL/well of Blocking Buffer (PBS and 10% FBS; Invitrogen, Carlsbad, Calif.) for two hours at room temperature. After washing, 75 μL of Assay Buffer (PBS, 10% FBS, 0.05% Tween-20, and HBRII) were added to each well followed by 25 μL of sample. The plates were incubated overnight at 4° C. and washed. One hundred μL of biotinylated mAb (1 μg/mL diluted in Blocking Buffer) were added to each well, and the plates were incubated for one hour at room temperature. After washing, 100 μL of horseradish peroxidase-conjugated streptavidin (BD) at a 1:1,000 dilution were added to each well, and the plates were incubated for 30 minutes at room temperature. The plates were washed and developed with TMB (Pierce Biotechnology, Rockford, Ill.). The reaction was stopped using 100 μL/well of 0.5 NH₂SO₄ and the plates were read at 450 nm using a Benchmark Plus plate reader (Biorad, Hercules, Calif.). For calibration of each sandwich ELISA, standards of 100 to 0.8 ng/mL of recombinant B7-H1 fusion polypeptide were analyzed in parallel with the test samples. The minimal detectable concentration (MDC) for the assay was determined to be 1 ng/mL.

### Example 3

### Detection of B7-H1 Polypeptide in the Serum of Patients with RCC

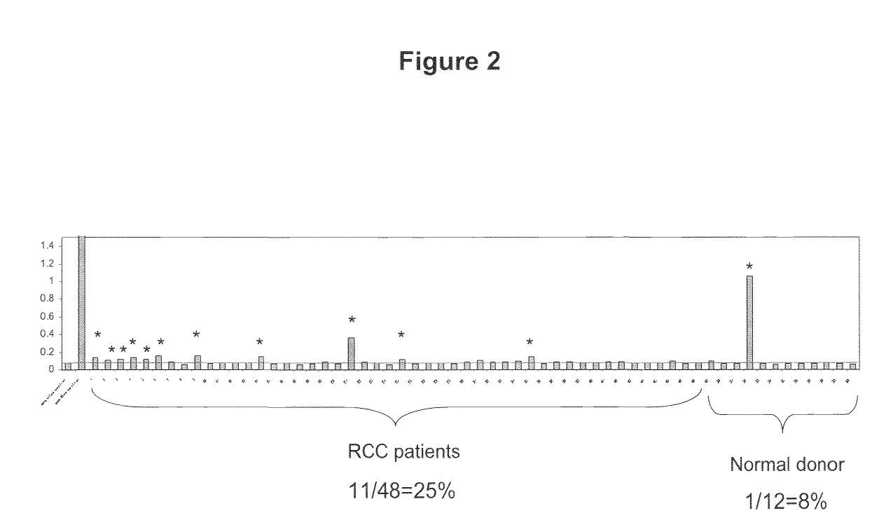

All serum samples were collected by venipuncture technique from patients and donors with appropriate informed consent. In general, blood collected by venipuncture was allowed to clot for 20 minutes at room temperature and then centrifuged for 15 minutes at 3,200 rpm. RCC cancer serum samples were collected before the surgical removal of the cancer, and stage and histology of the cancer were determined by pathologists. Normal controls were collected from healthy volunteers undergoing blood donation at blood transfusion center. The serum samples for the preliminary validation study included 12 normal controls (male and female) and 48 samples from patients with RCC. As shown in FIG. 2, eleven of 48 sera specimens from RCC patients demonstrated positive reactivity, while only one in 12 sera specimens from normal donors was positive.

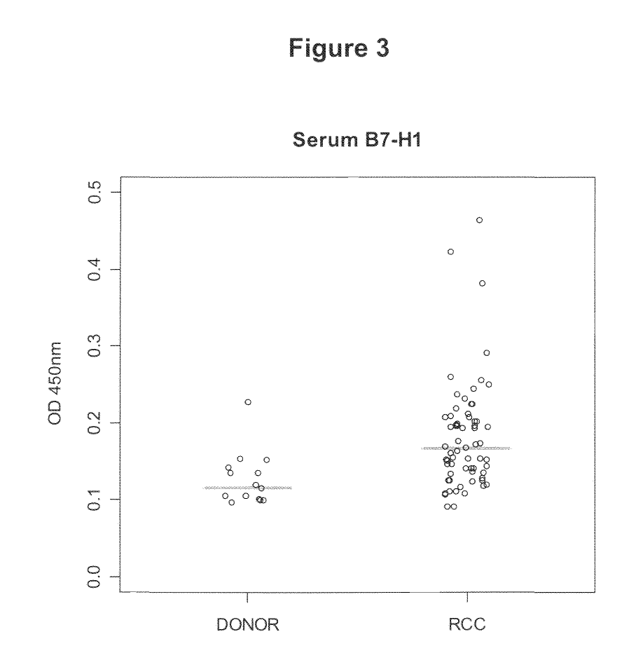

In another experiment, serum samples from 14 normal donors and 65 clear cell renal cell carcinoma patients were analyzed by sandwich ELISA with anti-B7-H1 polypeptide antibodies. The data were analyzed using the Wilcoxon rank sum test, and the p-value was 0.0008 (FIG. 3).

### Example 4

### Evaluation of Anti-B7-H1 Monoclonal Antibodies

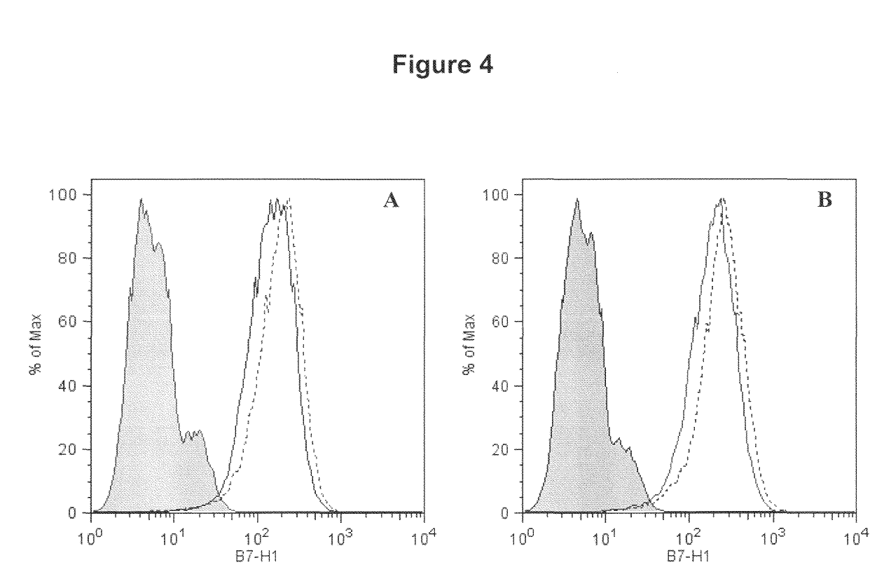

Two clones of monoclonal anti-B7-H1 antibodies, 2.2B and 5H1-A3, were created as described in Example 1. Experiments were performed to determine whether the 2.2B and 5H1-A3 antibodies recognize different epitopes of the same polypeptide. Cells expressing B7-H1 polypeptide were stained with biotinylated 2.2B antibody alone, or incubated with unbiotinylated 5H1-A3 antibody and then stained with biotinylated 2.2B antibody. In addition, cells expressing B7-H1 polypeptide were stained with biotinylated 5H1-A3 antibody alone, or incubated with unbiotinylated 2.2B antibody and stained with biotinylated 5H1-A3 antibody. The stained cells along with unbiotinylated control cells were analyzed using flow cytometry. Staining of cells with biotinylated 2.2B antibody in the absence of unbiotinylated 5H1-A3 antibody was comparable to staining in the presence of unbiotinylated 5H1-A3 antibody (FIG. 4A). Likewise, staining of cells with biotinylated 5H1-A3 antibody was comparable in the presence and absence of unbiotinylated 2.2B antibody (FIG. 4B). Results of these experiments indicate that the 2.2B and 5H1-A3 monoclonal anti-B7-H1 antibodies are directed against distinct epitopes of B7-H1 polypeptide.

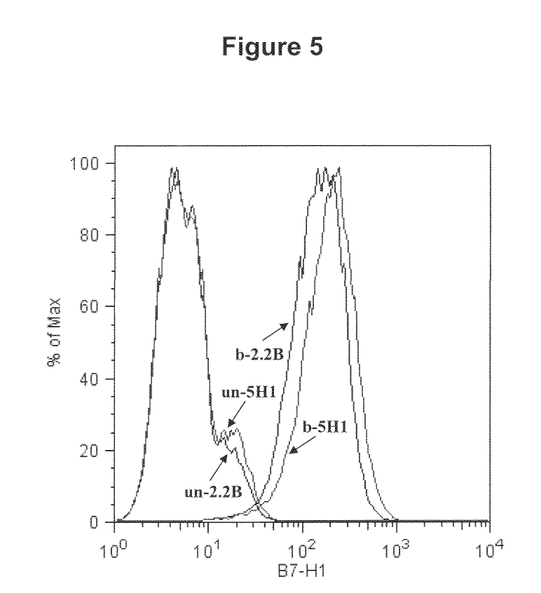

The intensity of staining produced by the 5H1-A3 antibody was compared to the intensity of staining produced by the 2.2B antibody. Cells expressing B7-H1 polypeptide were stained using biotinylated 5H1-A3 or 2.2B antibody. The stained cells, along with unbiotinylated control cells, were analyzed using flow cytometry. The intensity of staining produced using the 5H1-A3 antibody was observed to be comparable to the intensity of staining produced using the 2.2B antibody (FIG. 5).

### Example 5

### Detection of B7-H1 Polypeptide in Cell Culture Supernatants

Cell culture supernatants were analyzed for soluble B7-H1 polypeptide levels using an ELISA assay (see Example 2). Monoclonal anti-B7-H1 antibody 2.2B (2 μg/mL) was used as a capture antibody, and biotinylated monoclonal anti-B7-H1 antibody 5H1-A3 was used as a detection antibody. The supernatants consisted of cell culture media that had been incubated with cells for three to four days. Supernatants from the following cell lines were analyzed: a primary human bladder tumor cell line (BT10B), a human mastocyte cell line (HMCI), and a human kidney cancer cell line (Caki-2). Recombinant human B7-H1 fusion polypeptide, B7-H1.huFc, also referred to as B7H1Fc, was prepared as described in Example 1 and used as a positive control at a concentration of 1 μg/mL. A fusion polypeptide comprising the extracellular domain of a human or mouse P-selectin polypeptide fused to the CH2-CH3 domain of a human immunoglobulin G (PSelFc; see, e.g., catalog number 555294, BD Pharmingen™, BD, Franklin Lakes, N.J.) was used as a negative control at a concentration of 1 μg/mL. Phosphate-buffered saline (PBS) and cell culture media for BT10B and HMCI cells, which had not been contacted with cells, served as additional negative controls.

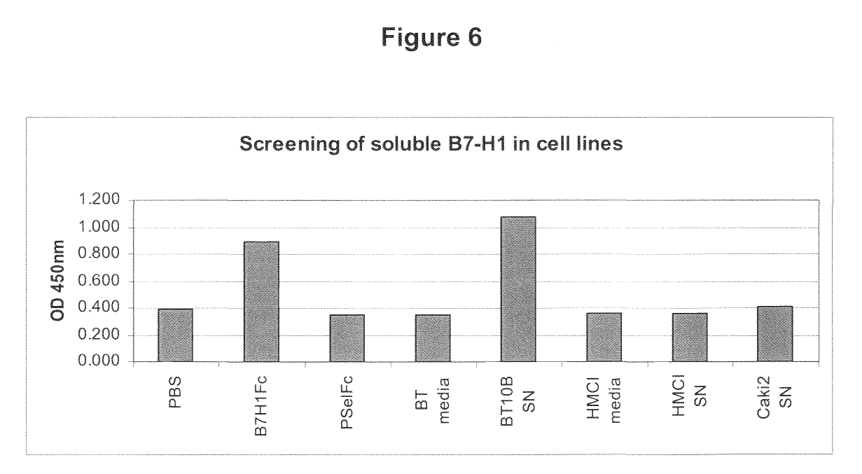

Results of these experiments indicated that BT10B cell supernatant was positive for B7-H1 polypeptide, whereas BT10B cell culture medium that had not been incubated with cells was negative (FIG. 6).

Additional experiments were performed to analyze B7-H1 polypeptide levels in cell culture supernatants from 624MEL, B7-H1/624MEL, J82, Caki-2, BT10B, and BT10C cells. 624MEL is a human melanoma cell line, B7H1/624MEL is a human melanoma cell line (624MEL) transfected with a B7-H1 polypeptide expression vector, J82 is a human bladder carcinoma cell line, Caki-2 is a human kidney cancer cell line, and BT10B and BT10C are primary human bladder tumor cell lines. B7-H1 polypeptide levels were analyzed using an ELISA assay, as described above. Recombinant human B7-H1 fusion polypeptide (B7H1Fc; 1 μg/mL) was used as a positive control, and the cell culture media for each cell line, along with PBS, were analyzed as negative controls.

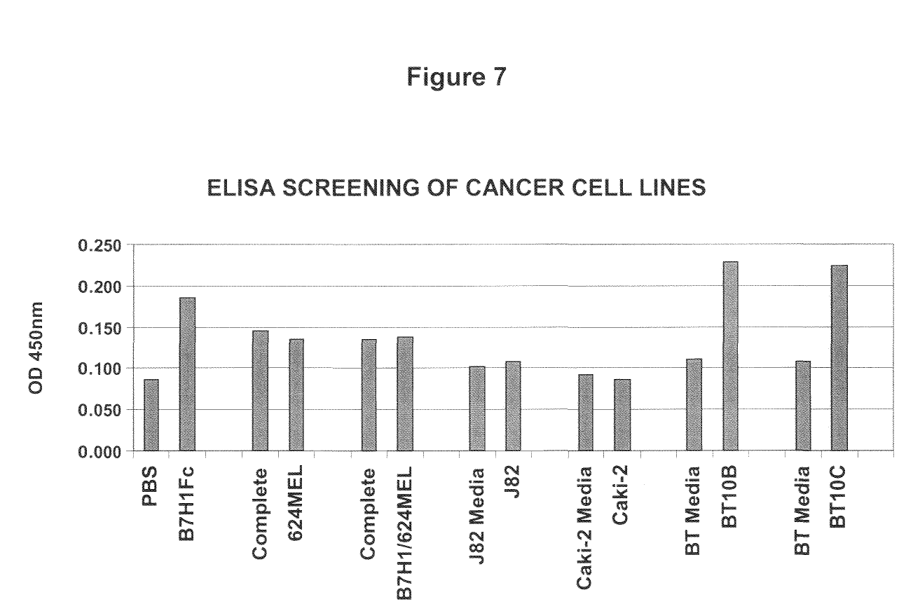

Results of these experiments indicated that supernatants from BT10B and BT10C cells were positive for B7-H1 polypeptide, whereas BT10B and BT10C cell culture media that had not been incubated with cells were negative (FIG. 7).

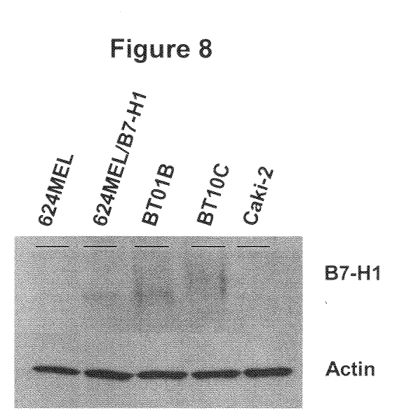

Extracts from 624MEL, B7-H1/624MEL, BT10B, BT10C, and Caki-2 cells were analyzed for B7-H1 polypeptide levels by Western blotting. Polypeptide extracts (50 μg) from each cell line were separated using gel electrophoresis, transferred to a membrane, and immunoblotted with a biotinylated 5H1-A3 antibody. The membrane was stripped and reprobed for actin polypeptide as a loading control. B7-H1 polypeptide was detected in extracts from B7-H1/624MEL, BT10B, and BT10C cells, but not in extracts from 624MEL or Caki-2 cells (FIG. 8).

### Example 6

### Materials and Methods for Examples 7 to 10

sB7-H1 sandwich ELISA: 2.2B was used as the plate-fixed capture antibody and biotinylated 5H1-A3 as the detection antibody. Biotinylation was performed using a solid-phase kit (Pierce; Rockford, Ill.). After each step in the assay, the plates were washed three times in washing buffer (PBS+0.05% Tween-20) using an automatic plate washer (Bio-Tek; Winooski, Vt.). High-binding polystyrene plates (Corning Life Sciences; Lowell, Mass.) were coated overnight at 4° C. with 0.1 μg/well of 2.2B. Any remaining coating solution was then aspirated, the wells were washed, and any free Fc binding sites were blocked with 200 μL/well of blocking buffer (PBS+10% FBS) for 2 hours at 21° C. After washing, 75 μL of assay buffer (PBS+10% FBS+0.05% Tween-20+HBR-I) were added to each well followed by 25 μL of sample serum and incubated overnight at 4° C. HBR-J (Scantibodies; Santee, Calif.) was added to prevent the non-specific binding of heterophilic antibodies. The plates were washed, and 100 μL of biotinylated 5H1-A3 (1 μg/mL diluted in blocking buffer) were added to each well and incubated for 1 hour at 21° C. After washing, 100 μL of horseradish peroxidase-conjugated streptavidin (BD Biosciences; San Jose, Calif.) were added to each well and incubated for 30 minutes at 211 C. The plates were washed and developed with TMB (Pierce). Reactions were stopped using 100 μL/well of 0.5N H₂SO₄ and the plates were read at 450 nm using a Benchmark Plus plate reader and associated software (Bio-Rad Laboratories; Hercules, Calif.). To calibrate each ELISA and obtain a standard detection curve, each plate contained parallel dilutions of recombinant B7-H1 fusion protein ranging in concentration from 0.8 to 100 ng/mL.

Capture and Detection Antibodies for Soluble B7-H1 (sB7-H1) ELISA: The hybridoma 5H1-A3 was subcloned from the reported anti-B7-H1-producing 5H1 hybridoma line (Dong et al. (2002) Nat. Med. 8:793-800). To generate the second (capture) monoclonal antibody, 2.2B, melanoma (624MEL) cells transfected with human full-length B7-H1 were injected intraperitoneally into 7 to 8-week-old Balb/c mice (5×10⁶ cells/injection) once per week for 6 weeks. Splenocytes were then isolated and fused with A38 cells to form a hybridoma using standard techniques (de StGroth and Scheidegger (1980) J. Immunol. Methods 35: 1-21). Both 5H1-A3 and 2.2B hybridoma supernatants were screened by ELISA for reactivity against the recombinant human protein B7-H1-human IgG (R&D Systems; Minneapolis, Minn.) and for the absence of cross-reactivity to an irrelevant recombinant protein P-Selectin-human IgG (BD Biosciences).

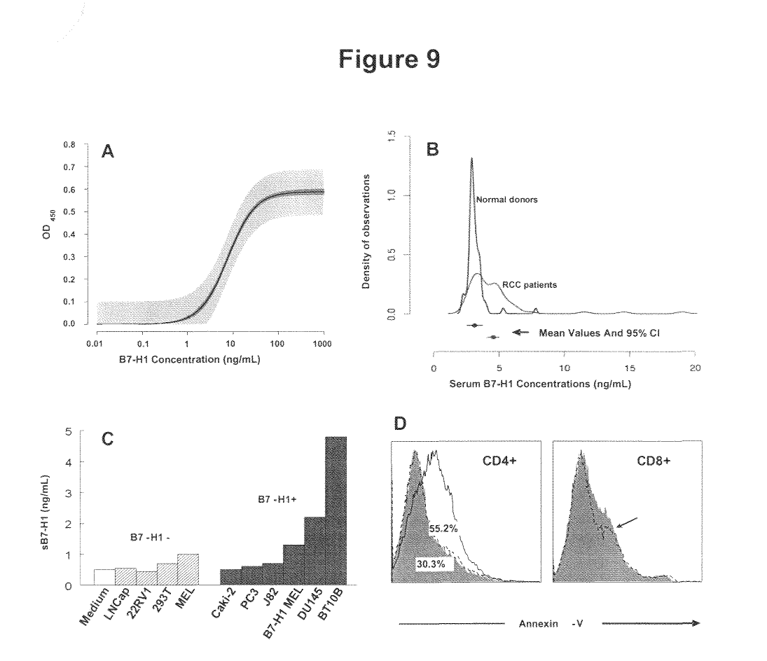

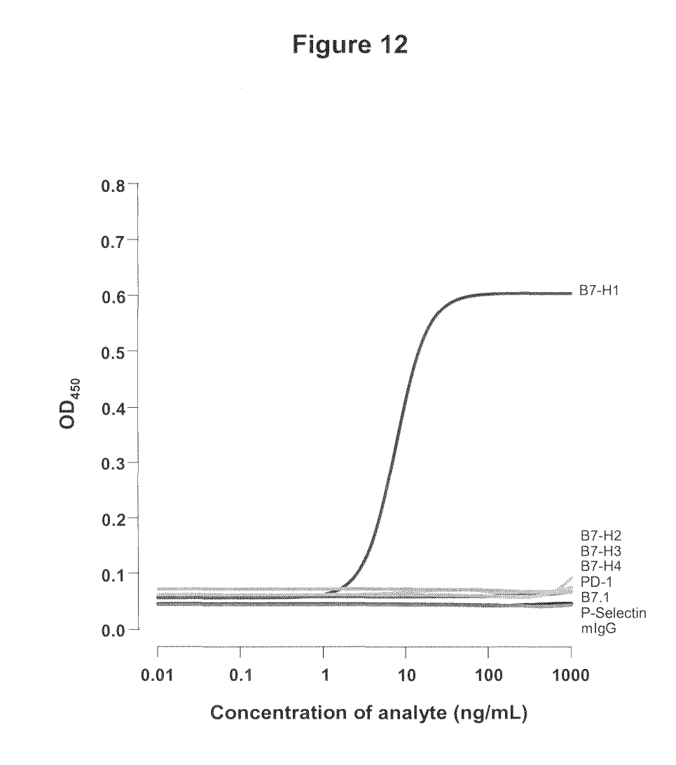

ELISA Calibration and Specificity Assessment: Calibration of the sandwich ELISA against standard B7-H1 dilution curves was done by fitting a 4-parameter logistic regression model using the drc package for R (Ritz and Streibig (2005) J. Statist. Software 12:22; and Robison-Cox (1995) J. Immunol. Methods 186:79-88). To measure the performance of the ELISA, a calibration plot with its 95% confidence (95% CI) and prediction intervals (95% PI) was generated (FIG. 9A) using 16 consecutive and independent assay runs done over a period of several weeks with quantities of B7-H1 fusion protein ranging from 1.2 pg/mL to 10 μg/mL. This calibration model showed a coefficient of determination (R²) of 0.959, demonstrating an excellent model fit and acceptably small interassay variability. The effective concentrations (EC) EC2.5, EC50, and EC97.5 were 0.6 ng/mL, 7.5 ng/mL and 98.6 ng/mL, respectively. Plotting the interassay coefficient of variation (CV) against the B7-H1 concentration standards showed that the variability between ELISAs was highest at the lowest concentrations of B7-H1. The interassay CV stabilized at approximately 10% when the B7-H1 concentration was within the range of EC2.5 to EC97.5, suggesting a lower and upper limit of quantification for the assay of approximately 1 ng/mL and 100 ng/mL, respectively. Specificity experiments for the B7-H1 ELISA are shown in FIG. 12. The purified proteins that were tested included B7-H2, B7-H3, B7-H4, B7.1 (CD80), and PD-1, which are similar in structure and function to B7-H1, as well as “nonsense” protein P-Selectin (R&D Systems), and non-specific murine IgG (BD Biosciences). Using generalized additive regression models constructed with the mgcv package for R (Wood (2006) “Generalized Additive Models: An Introduction with R.” (Chapman & Hall/CRC)), smooth fits of the ELISA results for the non-specific proteins were plotted and overlaid.

Cell Lines and Primary ccRCC Specimens: Cell lines 624MEL, 293T, Jurkat, Caki-2, J82, LNCaP, 22RV1, PC3 and DU145 were purchased and propagated per recommendations provided by the supplier (ATCC; Manassas, Va.). The B7-H1+624MEL cell line used in these studies was generated by transfecting a plasmid containing the full-length human B7-H1 sequence into the melanoma B7-H1-deficient 624MEL as previously described (Dong et al. (2002) Nat. Med. 8:793-800). BT10B is a spontaneously immortalized bladder cancer cell line that was established from a radical cystectomy specimen harboring high grade urothelial carcinoma. BT10B was established in 2004 and had been passaged >170 times at the time of the present experiments. This cell line expresses the markers uroplakin III and cytokeratin-20, indicative of urothelial origin (Parker et al. (2003) Am. J. Surg. Pathol. 27: 1-10). Primary RCC tumor cells were isolated from a 1-cm to 10-cm specimen of histologically-confirmed ccRCC tumor tissue obtained intraoperatively using sterile technique on kidney removal. Tissue was enzymatically dissociated in digestion medium as previously described (Siddiqui et al. (2007) Clin. Cancer Res. 13:2075-2081). Cells were then washed in PBS+5% FBS and centrifuged through a sucrose density gradient (Lymphoprep, Accurate Chemical and Scientific Corp). The buffy coat layer, representing the tumor infiltrating lymphocyte population, was discarded and the enriched primary RCC tumor cells were collected, washed twice with PBS+5% FBS, counted and cryopreserved at −80° C. for later processing.

Review of ccRCC Tumor Specimens for Cytoplasmic B7-H1 Expression:

Patient Selection and Features Studied -634 consecutive patients were identified from the Mayo Clinic Nephrectomy Registry that had been treated with radical nephrectomy or nephron-sparing surgery for unilateral, sporadic, non-cystic, ccRCC between 1990 and 1999. The clinical features studied included age, gender, symptoms at presentation, and Eastern Cooperative Oncology Group (ECOG) performance status. Patients with a palpable flank or abdominal mass, discomfort, gross hematuria, acute onset varicocele, or constitutional symptoms including rash, sweats, weight loss, fatigue, early satiety, and anorexia were considered symptomatic at presentation. The pathologic features studied included histologic subtype, the 2002 primary tumor classification, regional lymph node involvement, distant metastases, the 2002 TNM stage groupings, tumor size, nuclear grade, and coagulative tumor necrosis. Disease status for patients in the Nephrectomy Registry was updated each year. If a patient had not been seen in the previous year, the patient was sent a disease status questionnaire. If there was evidence of disease progression in this questionnaire, the date, location, and treatment were verified in writing with the patient's local physician. Patient vital status was similarly updated on a yearly basis. If a patient died in the previous year, a death certificate was ordered to determine the cause of death. A physician visit within six months of the date of death for metastatic RCC was considered good documentation that RCC was the cause of death. If the death certificate did not support this conclusion, the medical history was reviewed by a urologist to determine the cause of death. If a death certificate could not be obtained, the cause of death was be verified with the patient's family or local physician.

Patient Follow-up—At last follow-up, 359 patients had died, including 211 who died from RCC at an average of 3.3 years following surgery (median 2.1; range 0.1-14.0). Among the 275 patients who were still alive, the average duration of follow-up was 10.4 years (median 10.3; range 0.1-17.2); only 9 (3.3%) patients had fewer than two years of follow-up. Estimated cancer-specific survival rates (standard error [SE], number still at risk) at 5 and 10 years following surgery were 73.1% (1.8%, 394) and 64.4% (2.1%, 190), respectively.

Human Sera and Cancer Cell Media For sB7-H1 Analysis: Preoperative blood samples from cancer patients were collected by venipuncture. Samples were allowed to clot for 20 minutes at 21° C. and then centrifuged for 15 minutes at 3,200 rpm. A total of 58 ccRCC patients (45 male, 13 female) consented to the study, with a median age of 61.5 years (range 28-83). Normal control specimens were collected from healthy volunteers undergoing blood donation, and included 46 males and 33 females, with a median age of 68 years (range 17-87). A total of 19 patients (14 male, 5 female) with pancreatic cancer consented to the study, with a median age of 62 years (range 54-81). Normal controls included 70 males and 25 females, with a median age of 62 years (range 55-83). All samples were aliquoted upon arrival and stored at −80° C. until use. To perform the ELISA, serum samples were thawed on ice and three replicates were run along with protein standards. To minimize the impact of variance between ELISA plates, samples from cancer patients and control patients were run together on each 96 well plate. To test for a released form of sB7-H1 from human cancer cell lines, cells were typically cultured for three to five days, when aliquots of media were collected and centrifuged at 2000 g for 10 minutes to eliminate cellular debris, and then mixed with assay buffer as described above.

Apoptosis Assay for Activated Human T Cells: PBMCs were isolated from 11 Leukotrap W B leukoreduction filters (Pall Corp.; East Hills, N.Y.) used in the routine processing of normal human donor blood as previously described (Inman et al. (2008) J. Immunol. 180:3578-3584). CD4+ and CD8+ cells were obtained by negative magnetic isolation (Miltenyi Biotech; Auburn, Calif.). Cells were placed in 96-well plates pre-coated with 2 μg/mL of anti-human CD3 (UCHT1, BD Biosciences) and cultured for 3 days in complete media (RPMI+10% FBS 20 mM HEPES and Penicillin/Streptomycin). 10 μg/mL of azide-free recombinant human B7-H1-Fc or P-Selectin-Fc fusion protein (R&D Systems) were added to the activated T cell cultures and incubated overnight. Cells were then harvested and stained for Annexin V (BD Biosciences) and PI (Sigma; St. Louis, Mo.), and relative percentages of apoptotic (Annexin V+PI−) CD4+ and CD8+ T cells were quantified by flow cytometry using a FACSCalibur flow cytometer (BD) and analyzed with FlowJo software (Tree Star; Ashland, Oreg.).

Cloning of the sB7-H1 Splice-Variant: The publicly available Uniprot database (World Wide Web at pir.uniprot.org), which contains sequence information for proteins and their corresponding mRNAs, was searched for TM-deficient forms of human B7-H1. Protein alignment was performed using commercially available software (MacVector 8.0; MacVector, Inc.; Cary, N.C.). The identified sequence Q9NZQ7-3 (referred to herein asB7-H1^{Δ531-636}) was then studied in silico at the mRNA level. Exonic sequences for B7-H1 and sB7-H1^{Δ531-636} were obtained from the Ensembl genome browser (World Wide Web at ensembl.org) and aligned using multiple sequence alignment (Corpet (1988) Nucl. Acids Res. 16:10881-10890). For RT-PCR experiments, messenger RNA was extracted from cell lines or primary RCC cells using the mRNeasy kit (Qiagen) and reverse transcription was performed with the iScript cDNA Synthesis kit (Bio-Rad), all according to manufacturer's instructions. Primers were then designed (IDT; Coralville, Iowa) to amplify both full-length B7-H1 and sB7-H1^{Δ531-636}. These primers targeted flanking regions of the predicted deletion, i.e., exon 3 (p1 forward, 5′-TACTGTCACGGTTCCCAAGG-3′; SEQ ID NO:3) and the conserved region in exon 5 (p2 reverse, 5′-ATTTGGAGGATGTGCCAGAG-3′; SEQ ID NO: 4), and resulted in an amplicon having an expected size of 651 bp for B7-H1 and 547 bp for sB7-H1^{Δ531-636}. PCR was performed using a high-fidelity Pfx DNA polymerase (Invitrogen; Carlsbad, Calif.) over 35 cycles in a standard thermocycler (iCycler, Bio-Rad). The RT-PCR amplification products were excised from an agarose gel, purified using gel extraction spin columns (Bio-Rad), and cloned into a pCR-BluntII-TOPO vector (Invitrogen). DNA sequencing confirmed the hypothesized A531-636 deletion. A third primer (p3 reverse 5′-CCTCAGGATCTAATCTCCACTCA-3′; SEQ ID NO:5) was designed against the splice point region. When p1 and p3 were combined, a RT-PCR band of 490 bp was obtained, which was specific for sB7-H1^{Δ531-636}. These RT-PCR products were cloned and sequenced as described above. RT-PCR with β-actin specific primers was performed to control for DNA loading (β-actin forward: 5′-TGACGGGGTCACCCACACTGTGCCCATCTA-3′ (SEQ ID NO:6); β-actin reverse: 5′-CTAGAAGCATTTGCGGTGGACGATGGAGGG-3′ (SEQ ID NO:7)).

Review of ccRCC Tumors for Cytoplasmic-Only B7-H1+ Expression: Specimens from 634 consecutive ccRCC patients treated with nephrectomy for ccRCC between 1990 and 1999 were examined for tumor cell patterns of IHC B7-H1 expression. IHC staining for B7-H1 expression was performed as previously reported (Thompson et al. (2006) Cancer Res. 66:3381-3385). All slides were scored by a single pathologist using traditional criteria (i.e., tumor cells exhibiting both membranous and cytoplasmic B7-H1+ staining) to quantify percentages of B7-H1+ tumor cells in each specimen at 5-10% increments. Subsequently, all B7-H1+ ccRCC tumors were rescored by the same pathologist, who recorded relative amounts of tumor cells exhibiting a cytoplasmic-only pattern of B7-H1+ staining, expressed as a subset percentage of total B7-H1+ tumor cells within a given ccRCC specimen. Multiple other human malignancies also were examined for tumor cells that exclusively express cytosolic but not membranous B7-H1.

Statistical Analyses: Wilcoxon rank-sum tests were used to compare sB7-H1 levels between cancer patients and normal donors. 95% confidence intervals (CI) for the mean B7-H1 levels were computed from 10,000 bootstrap samples. A kernel density plot using a Gaussian kernel function and the smoothing bandwidth of Sheather-Jones is presented to contrast the distribution of serum B7-H1 in cancer patients with that of normal donors (Sheather (2004) Statistical Sci. 19:588-597). A paired t-test was used to compare the effect of solubilized B7-H1-Fc or P-Selectin-Fc in activated T cells. All P-values were two-sided and considered statistically significant if <0.05. Additional statistical analyses to calibrate and validate the B7-H1 ELISA assay were performed as described herein.

Associations of tumor B7-H1 expression with clinical and pathologic features were evaluated using chi-square tests. Associations of tumor B7-H1 expression with cancer-specific survival were depicted using Kaplan-Meier plots, while the magnitude of such associations were evaluated using Cox proportional hazards regression models and summarized with risk ratios and 95% CIs. For these analyses, patients were categorized into three groups based on tumor expression of B7-H1: (1) those with B7-H1−tumors; (2) those with B7-H1+ tumors that were exclusively composed of tumor cells exhibiting combined membranous and cytoplasmic staining profiles; and (3) those with B7-H1+ tumors that harbored subsets of tumor cells lacking any membranous B7-H1+ expression and exhibiting only cytoplasmic B7-H1+ expression.

### Example 7

### ELISA Measurement of sB7-H1 in the Sera of Cancer Patients and Media of Human Cancer Cell Lines

To determine whether soluble forms of B7-H1 appear in the sera of cancer patients, an ELISA was developed that utilizes a pair of monoclonal antibodies raised against human extracellular B7-H1 (as described above). Monoclonal antibodies 2.2B and 5H1-A3 (both mouse IgG1) were used as capture and detection antibodies, respectively, to establish the ELISA. Using recombinant hB7-H1-Fc fusion protein, this ELISA exhibits an optimum detection range from 1 to 100 ng/mL, and a coefficient of variation of 10% within this range (FIG. 9A). Moreover, seven other related or control proteins (B7-H2, B7-H3, B7-H4, B7.1, PD-1, non-specific murine IgG, and P-Selectin) failed to exhibit measurable cross-reactivity, thus supporting the specificity of the assay (FIG. 12).

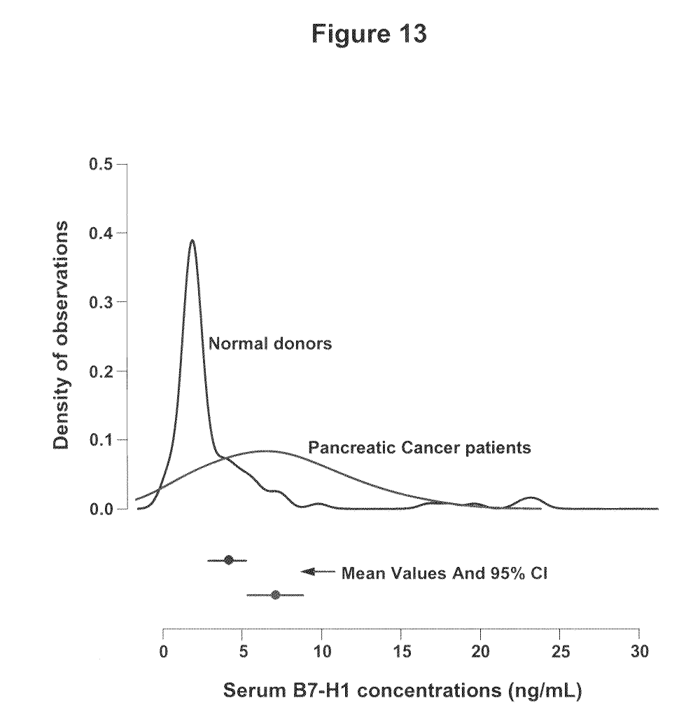

Using this ELISA, sera from ccRCC and pancreatic cancer patients were assayed for levels of sB7-H1 relative to sera from non-cancer control patients. FIG. 9B demonstrates that sera from ccRCC patients (n=58) exhibited significantly higher concentrations of sB7-H1 compared to sera from non-cancer controls (n=79; p<0.001), although some overlap in levels of sB7-H1 was observed between the two groups. In a separate pilot study, sB7-H1 levels in sera from pancreatic cancer patients were observed to be significantly higher than non-cancer controls (FIG. 13). Thus, serum levels of sB7-H1 in cancer patients were generally higher than non-cancer control patients. To further establish that B7-H1-expressing tumor cells can release sB7-H1, the media of B7-H1+ and B7-H1−human cancer cell lines was screened. FIG. 9C shows that sB7-H1 was released by the B7-H1+ bladder cancer cell line, BT10B, and the B7-H1+ prostate cancer cell line, DU145. Other B7-H1+ cell lines (B7-H1/624MEL, Caki-2, J82 and PC3) and B7-H1−cell lines (624MEL, 293T, Jurkat, LNCaP and 22RV1) failed to release detectable levels of sB7-H1 into the media (FIG. 9C).

### Example 8

### Effect of sB7-H1 on Activated CD4+ and CD8+ T Cell Apoptosis

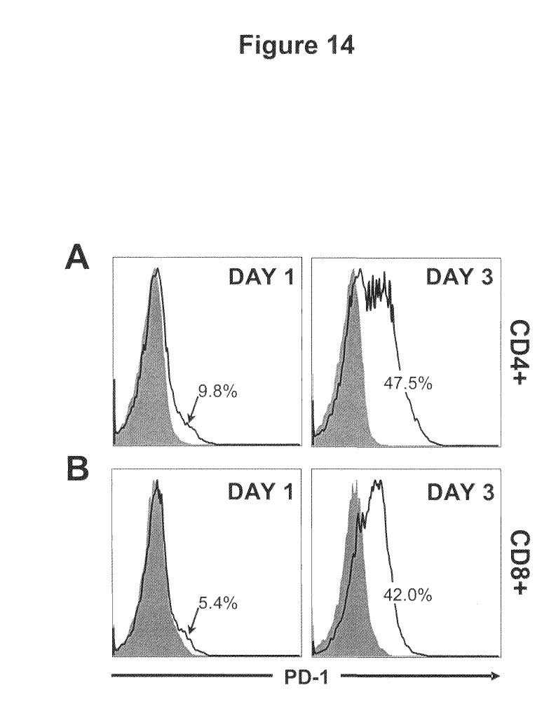

Membrane-bound B7-H1 has been reported to enhance apoptosis of activated T cells in murine systems (Dong et al. (2002) Nat. Med. 8:793-800; and Hori et al. (2006) J. Immunol. 177:5928-5935). Experiments were conducted to assess whether unbound (soluble) B7-H1 might enhance human T cell apoptosis. For these experiments, bead-purified CD4+ or CD8+ T cells were activated with anti-CD3 for 3 days. Activation was confirmed by surface expression of PD-1, a cognate receptor of B7-H1 (Freeman et al. (2000) J. Exp. Med. 192:1027-1034) (FIG. 14). Pre-activated CD4+ or CD8+ T cells were then incubated in the presence of either solubilized B7-H1-Fc or control P-Selectin-Fc fusion protein for 16 hours and analyzed for apoptosis based on Annexin V and propidium iodide (PI) staining. Exposure to soluble B7-H1-Fc enhanced apoptosis of pre-activated CD4+ T cells (p=0.019; FIG. 9D, left panel) to a greater extent than CD8+ T cells (p=0.899; FIG. 9D, right panel), whereas, soluble P-Selectin-Fc failed to alter rates of apoptosis for either population (FIG. 9D, both panels). Based on these results, it can be inferred that tumor-secreted soluble forms of B7-H1 affect activated CD4+ T cells, perhaps to impair systemic immunity.

### Example 9

### Identification of sB7-H1^{Δ531-636} as a New Splice Variant in Human Tumor Cell Lines

Full-length B7-H1 is a 290 amino acid protein that is comprised of signal peptide, Ig-V ligand-binding, Ig-C structural, transmembrane (TM) and intracellular domains (Dong et al. (1999) Nat. Med. 5:1365-1369). Since loss of the TM domain could cause B7-H1 ligand to be freely released as a soluble protein by cells, the Uniprot protein database was searched for TM domain-deficient forms of B7-H1. This search revealed one such form of B7-H1, sequence Q9NZQ7-3 (also named B7-H1 splice-variant II) which had not been previously reported in the literature. Alignment of Q9NZQ7-3 with full-length B7-H1 revealed truncation of C-terminal amino acids 179-290, resulting in partial loss of the Ig-C domain as well as complete loss of TM and intracellular domains (FIG. 10A). Thus, the splice-variant Q9NZQ7-3 sequence was investigated further as a candidate for sB7-H1.

An exon-by-exon comparison of B7-H1 mRNA sequences revealed that exon 4 (Ig-C domain) harbors a deletion of 106 nucleotides (A531-636, FIG. 10B) resulting in a frameshift and a de novo stop codon at position 179 that would halt transcription of the final 111 amino acids of full-length B7-H1, including its TM anchoring region. The Δ531-636 deletion also results in a K to D amino acid substitution at position 178 of B7-H1 (referred to as sB7-H1^{A531-636} hereafter) as illustrated in FIG. 10C. A separate downstream deletion in the TM exon 5 (A725-791) also was discovered, but it is functionally silenced by the upstream stop codon generated by the Δ531-636 deletion.

To test whether sB7-H1^{Δ531-636} is expressed by human cancer cells, RT-PCR was conducted using mRNA extracted from several of the aforementioned B7-H1+ and B7-H1− cell lines with specific PCR primers, p1 and p2, as described above. As predicted, RT-PCR yielded two PCR products from the two cancer cell lines noted to release measurable amounts of sB7-H1, namely BT10B and DU145 (FIG. 10D, top panel). The two bands were of anticipated sizes, corresponding to full-length B7-H1 (651 bp) and truncated sB7-H1^{Δ531-636} (547 bp). Sequencing of the bands confirmed that these PCR products were identical to canonical full-length B7-H1 and sB7-H1^{Δ531-636}. In contrast, Caki-2, which failed to release measurable amounts of sB7-H1, revealed strong full-length B7-H1 expression and little if any sB7-H1^{Δ531-636} expression (FIG. 10D, top panel). Additional RT-PCR employing a specific primer set (p1 and p3) designed to amplify only sB7-H1^{Δ531-636} from its splice point revealed strong expression of the anticipated 490 bp sB7-H1^{Δ531-636} PCR product by BT10B and DU145 cells, and little or no expression by Caki-2 cells (FIG. 10D, bottom panel).

### Example 10

### Accumulation of Cytoplasmic B7-H1 in Tumor Cells is Correlated with Poor Prognosis in RCC

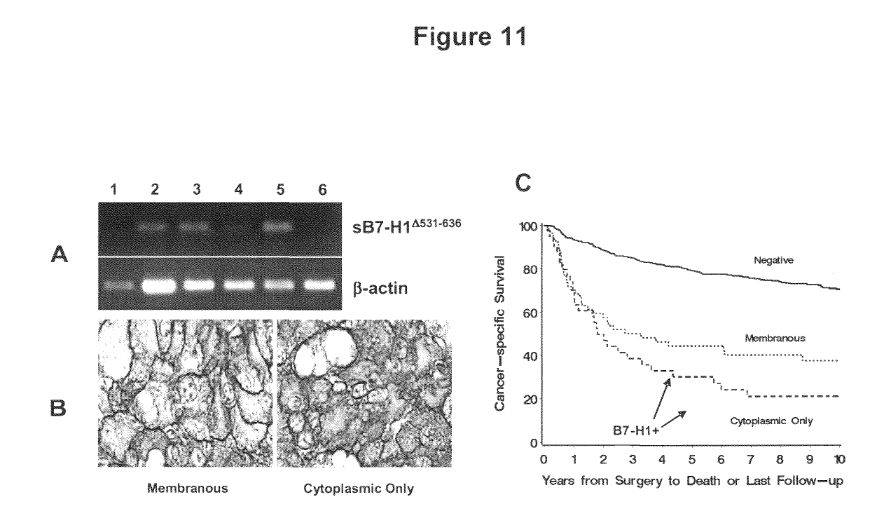

B7-H1 expression in tumor cells portends aggressive disease course and poor cancer-specific survival for patients with ccRCC (Thompson et al. (2004) Proc. Natl. Acad. Sci. USA 101:17174-17179; and Thompson et al. (2006) Cancer Res. 66:3381-3385). Little attention has been given, however, to specific cellular patterns of B7-H1 expression. In general, tumors exhibiting predominantly membranous (with or without cytoplasmic) staining have been scored as B7-H1+. The new finding that human cancer cells can express sB7-H1^{Δ531-636} (above), suggested the possibility that this splice-variant of B7-H1 might be expressed in ccRCC tumors as well. RT-PCR interrogation of tumor cells from fresh, enzyme-digested ccRCC specimens (FIG. 11A) revealed that some human ccRCC tumors do indeed express sB7-H1^{Δ531-636}.

Given that sB7-H1^{Δ531-636} possesses an intact signaling domain but lacks its TM anchor, it was surmised that IHC expression of this B7-H1 splice-variant might appear in tumor cells exhibiting cytoplasmic B7-H1 expression without attendant membranous staining. Thus, patterns of tumor cell B7-H1 staining were examined in specimens from 634 consecutive ccRCC patients treated with nephrectomy between 1990 and 1999. Additionally, patterns of B7-H1 staining were correlated with clinicopathologic features of disease and ccRCC patient outcomes. Of note, the anti-B7-H1 antibody used for the IHC studies (clone 5H1-A3) was originally generated against the extracellular domain of B7-H1 ligand and can therefore recognize both full-length B7-H1 and truncated sB7-H1.

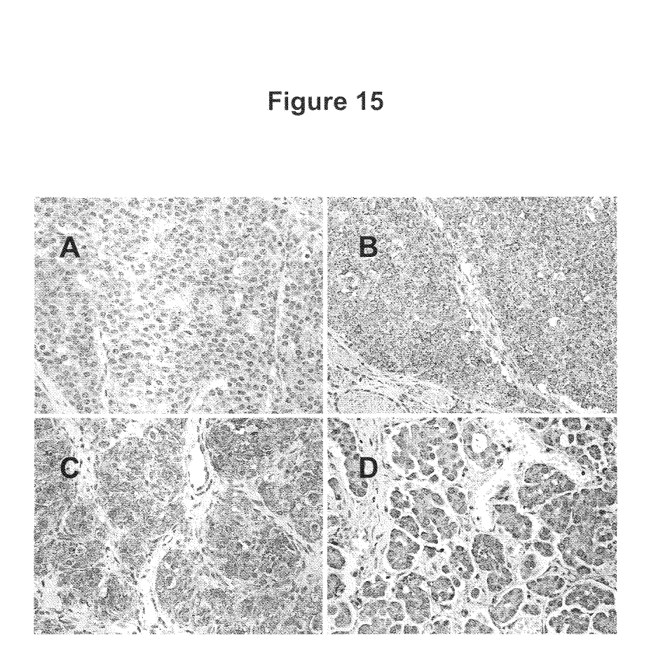

Of the 634 ccRCC tumor specimens studied, 97 (15.3%) were identified as B7-H1+. Fifty-seven (58.8%) of these B7-H1+ tumors were exclusively composed of tumor cells exhibiting combined membranous and cytoplasmic staining profiles (referred to as membranous B7-H1+ hereafter; FIG. 11B, left panel). In contrast, 40 (41.2%) of these B7-H1+ tumors also harbored subsets of tumor cells that lacked any membranous B7-H1+ expression, exhibiting “cytoplasmic-only” B7-H1+ expression (FIG. 11B, right panel). For the latter group, percentages of tumor cells exhibiting cytoplasmic-only B7-H1+ expression ranged from 2% to 70% of total B7-H1+ tumor cells observed. Cytoplasmic-only patterns of B7-H1+ staining also were observed in other malignancies or neoplasms, including neuroblastoma, medullary carcinoma of thyroid, paraganglioma and ovarian serous carcinoma (FIG. 15).

As shown in Table 1, nearly every index of severity of ccRCC disease increased with the presence of tumor cells exhibiting cytoplasmic-only B7-H1+. For instance, every tumor exhibiting this staining profile was a high grade (grade 3 or 4) malignancy (p<0.001). FIG. 11C illustrates that patients with membranous B7-H1+ tumors were 3.5 times more likely to die from RCC than patients whose tumors were B7-H1-(risk ratio 3.47; 95% CI 2.41-4.98; p<0.001); a finding fully consistent with previous reports (Thompson et al. (2004) Proc. Natl. Acad. Sci. USA 101:17174-17179; and Thompson et al. (2006) Cancer Res. 66:3381-3385). However, those patients with B7-H1+ tumors that contained subsets of cytoplasmic-only B7-H1+ tumor cells were nearly 5 times more likely to die from RCC than patients with B7-H1−tumors (risk ratio 4.90; 95% CI 3.28-7.33; p<0.001). Although the differences in cancer-specific survival for the two groups of patients with B7-H1+ tumors failed to achieve statistical significance in this study (p=0.216), it is clear that the presence of cytoplasmic-only B7-H1+ tumor cells delineates a particularly aggressive form of ccRCC. Finally, the cytoplasmic-only pattern of B7-H1+ expression that was observed further suggests the possibility of sB7-H1^{Δ531-636} expression by ccRCC tumors.

TABLE 1
Comparison of clinical and pathologic features
by B7-H1 expression for 634 patients with ccRCC

| | Tumor B7-H1 Expression | | | |
|---|---|---|---|---|
| | | Membranous | | |
| | Negative | N = 57 | Cytoplasmic-only | |
| Feature | N = 537 | N (%) | N = 40 | P-value |
| Age at Surgery (years) | | | | |
| <65 | 280 (52.1) | 28 (49.1) | 14 (35.0) | 0.108 |
| ≧65 | 257 (47.9) | 29 (50.9) | 26 (65.0) | |
| Gender | | | | |
| Female | 192 (35.8) | 18 (31.6) | 11 (27.5) | 0.493 |
| Male | 345 (64.2) | 39 (68.4) | 29 (72.5) | |
| Symptoms | | | | |
| Absent | 208 (38.7) | 14 (24.6) | 6 (15.0) | 0.002 |
| Present | 329 (61.3) | 43 (75.4) | 34 (85.0) | |
| Constitutional Symptoms | | | | |
| Absent | 421 (78.4) | 35 (61.4) | 16 (40.0) | <0.001 |
| Present | 116 (21.6) | 22 (38.6) | 24 (60.0) | |
| ECOG Performance Status | | | | |
| 0 | 484 (90.1) | 50 (87.7) | 38 (95.0) | 0.486 |
| ≧1 | 53 (9.9) | 7 (12.3) | 2 (5.0) | |
| 2002 Primary Tumor | | | | |
| Classification | | | | |
| pT1a | 154 (28.7) | 5 (8.8) | 4 (10.0) | <0.001 |
| pT1b | 164 (30.5) | 14 (24.6) | 4 (10.0) | |
| pT2 | 85 (15.8) | 10 (17.5) | 8 (20.0) | |
| pT3a | 48 (8.9) | 6 (10.5) | 5 (12.5) | |
| pT3b | 74 (13.8) | 20 (35.1) | 15 (37.5) | |
| pT3c | 7 (1.3) | 2 (3.5) | 1 (2.5) | |
| pT4 | 5 (0.9) | 0 | 3 (7.5) | |
| Regional Lymph Node | | | | |
| Involvement | | | | |
| pNX and pN0 | 521 (97.0) | 52 (91.2) | 34 (85.0) | <0.001 |
| pN1 and pN2 | 16 (3.0) | 5 (8.8) | 6 (15.0) | |
| Distant Metastases | | | | |
| pM0 | 493 (91.8) | 45 (79.0) | 26 (65.0) | <0.001 |
| pM1 | 44 (8.2) | 12 (21.0) | 14 (35.0) | |
| 2002 TNM Stage Groupings | | | | |
| I | 308 (57.4) | 17 (29.8) | 6 (15.0) | <0.001 |
| II | 73 (13.6) | 4 (7.0) | 4 (10.0) | |
| III | 106 (19.7) | 21 (36.8) | 15 (37.5) | |
| IV | 50 (9.3) | 15 (26.3) | 15 (37.5) | |
| Tumor Size (cm) | | | | |
| <5 | 199 (37.1) | 11 (19.3) | 6 (15.0) | <0.001 |
| ≧5 | 338 (62.9) | 46 (80.7) | 34 (85.0) | |
| Nuclear Grade | | | | |
| 1 | 43 (8.0) | 0 | 0 | <0.001 |
| 2 | 286 (53.3) | 7 (12.3) | 0 | |
| 3 | 184 (34.3) | 38 (66.7) | 18 (45.0) | |
| 4 | 24 (4.5) | 12 (21.1) | 22 (55.0) | |
| Coagulative Tumor | | | | |
| Necrosis | | | | |
| No | 422 (78.6) | 16 (28.1) | 4 (10.0) | <0.001 |
| Yes | 115 (21.4) | 41 (71.9) | 36 (90.0) | |
| UISS | | | | |
| I | 219 (40.8) | 5 (8.8) | 0 | <0.001 |
| II | 262 (48.8) | 35 (61.4) | 25 (62.5) | |
| III | 15 (2.8) | 2 (3.5) | 0 | |
| IV | 40 (7.5) | 14 (24.6) | 14 (35.0) | |
| V | 1 (0.2) | 1 (1.8) | 1 (2.5) | |
| SSIGN Score | | | | |
| 0-2 | 270 (50.3) | 8 (14.0) | 1 (2.5) | <0.001 |
| 3-6 | 177 (33.0) | 15 (26.3) | 8 (20.0) | |
| 7+ | 90 (16.7) | 34 (59.7) | 31 (77.5) | |

Membranous: B7-H1+ tumors that were exclusively composed of tumor cells exhibiting combined membranous and cytoplasmic staining profiles
Cytoplasmic Only: B7-H1+ tumors that harbored subsets of tumor cells that lacked any membranous B7-H1+ expression

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of evaluating a mammal, said method comprising (a) providing a body fluid from said mammal, and (b) detecting the presence or absence of B7-H1 in said body fluid.

2. The method of claim 1, wherein said mammal is a human.

3. The method of claim 1, wherein B7-H1 is detected immunologically.

4. The method of claim 1, wherein B7-H1 is detected using a monoclonal antibody.

5. The method of claim 1, wherein B7-H1 is detected using a capture antibody and a reporter antibody, wherein said reporter antibody comprises a label.

6. The method of claim 1, wherein said body fluid is selected from the group consisting of blood, plasma, serum, urine, cerebrospinal fluid, sputum, tears, and saliva.

7. The method of claim 6, wherein said body fluid is serum.

8. The method of claim 1, wherein said mammal is suspected of having a cancer.

9. The method of claim 8, wherein said cancer is renal cell carcinoma.

10. The method of claim 1, wherein the presence of B7-H1 in said body fluid indicates the presence of a cancer in said mammal.

11. The method of claim 10, wherein the presence of B7-H1 in said body fluid indicates said mammal is more likely to die of said cancer than if B7-H1 is absent.

12. A method of detecting B7-H1 in a body fluid, said method comprising:
(a) providing a solid substrate, said solid substrate coated with capture antibodies having binding affinity for soluble B7-H1;
(b) contacting said body fluid with said solid substrate under conditions in which soluble B7-H1, if present, becomes bound to said solid substrate to form a first reacted solid substrate;
(c) contacting said first reacted solid substrate with a reporter antibody having binding affinity for soluble B7-H1 to form a second reacted solid substrate; and
(d) detecting the presence or absence of said reporter antibody on said second reacted solid substrate, wherein the presence of reporter antibody indicates that soluble B7-H1 is present in said body fluid.

13. The method of claim 12, wherein said reporter antibody comprises a label selected from the group consisting of a radioisotope, a fluorophore, a luminescent moiety, biotin, and an enzyme.

14. The method of claim 12, wherein detecting the presence or absence of said reporter antibody comprises contacting said second reacted solid substrate with a secondary antibody having binding affinity for said reporter antibody, wherein said secondary antibody comprises a label.

15. The method of claim 12, wherein detecting the presence or absence of said reporter antibody comprises contacting said second reacted solid substrate with a reagent having binding affinity for said reporter antibody, wherein said reagent comprises a label.

16. The method of claim 12, wherein said solid substrate is a bead or a microtiter plate.

17. A method for determining whether a mammal has cancer, said method comprising (a) determining whether or not a mammal has a body fluid containing an elevated level of a B7-H1 polypeptide, and (b) classifying said mammal as having cancer if said mammal has said elevated level or classifying said mammal as not having cancer if said mammal does not have said elevated level.

18. The method of claim 17, wherein said mammal is a human.

19. The method of claim 17, wherein said body fluid is blood, serum, plasma, or urine.

20. The method of claim 17, wherein said cancer is renal cell carcinoma.


SEQUENCE LISTING (7 sequences)

SEQ ID NO: 1 - protein, 290 aa, Homo sapien
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 178 aa, Homo sapien
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 3 - DNA, 20 bp, Artificial Sequence
  Primer
tactgtcacggttcccaagg

SEQ ID NO: 4 - DNA, 20 bp, Artificial Sequence
  Primer
atttggaggatgtgccagag

SEQ ID NO: 5 - DNA, 23 bp, Artificial Sequence
  Primer
cctcaggatctaatctccactca

SEQ ID NO: 6 - DNA, 30 bp, Artificial Sequence
  Primer
tgacggggtcacccacactgtgcccatcta

SEQ ID NO: 7 - DNA, 30 bp, Artificial Sequence
  Primer
ctagaagcatttgcggtggacgatggaggg